(19) United States
(12) Patent Application Publication
(10) Pub. No.: US 2026/0256889 A1
(43) Pub. Date: Sep. 3, 2026

(54) Pharmaceutical formulations comprising a cyclodextrin

(71) Applicant: Novo Nordisk A/S, Bagsvaerd (DK)

(72) Inventors: Benjamin Troest Kjeldsen, Broenshoej (DK); Stig Christoffersen, Copenhagen (DK); Rosa Rebecca Erritzoee Hansen, Koebenhavn OE (DK)

(21) Appl. No.: 18/715,475

(22) PCT Filed: Dec. 13, 2022
(86) PCT No.: PCT/EP2022/085558

§ 371 Date: May 31, 2024

(30) Foreign Application Priority Data
Dec. 13, 2021 (EP) 21214004.0
Aug. 22, 2022 (EP) 22191564.8

(51) Int. Cl. A61K 38/26; A61K 38/22; A61K 47/22; A61K 47/26; A61K 47/40

(52) U.S. Cl. CPC A61K 38/26; A61K 38/22; A61K 47/22; A61K 47/26; A61K 47/40

(57) ABSTRACT

Disclosed herein is a liquid pharmaceutical formulation comprising an amylin receptor agonist, a GLP-1 receptor agonist and a cyclodextrin comprising hydroxypropyl substitutions. Said co-formulation may be used for the medical treatment of subjects with overweight or obesity, with or without associated co-morbidities; diabetes, with or without associated comorbidities; cardiovascular diseases, non-alcoholic steatohepatitis (NASH) and cognitive impairment, such as that caused by Alzheimer's disease.

### TECHNICAL FIELD

The current invention relates to a pharmaceutical formulation which is a co-formulation of a GLP-1 receptor agonist and an amylin receptor agonist. Said pharmaceutical formulation may be used for the medical treatment of subjects with overweight or obesity, with or without one or more associated co-morbidities; diabetes, with or without one or more associated comorbidities; one or more cardiovascular diseases; non-alcoholic steatohepatitis (NASH); and/or cognitive impairment, such as that caused by Alzheimer's disease.

### BACKGROUND

Semaglutide is a glucagon-like peptide 1 (GLP-1) receptor agonist and is the active pharmaceutical ingredient in Ozempic®. Ozempic® is indicated (i) as an adjunct to diet and exercise to improve glycemic control in adults with type 2 diabetes mellitus and (ii) to reduce the risk of major adverse cardiovascular events in adults with type 2 diabetes mellitus and established cardiovascular disease.

Semaglutide is also the active pharmaceutical ingredient in Wegovy®. Wegovy® is indicated as an adjunct to a reduced calorie diet and increased physical activity for chronic weight management in adult patients with an initial body mass index (BMI) of greater or equal to 30 kg/m² or greater than 27 kg/m², in the presence of at least one weight-related comorbidity.

Ozempic® and Wegovy® are liquid pharmaceutical formulations comprising 8 mM phosphate and having a pH of about 7.4.

A fixed-dose combination of an amylin receptor agonist, cagrilintide, and the GLP-1 receptor agonist, semaglutide, has been investigated for the treatment of overweight and obesity (Lancet 2021; 397:1736-48). The drug products investigated were in the form of separate liquid pharmaceutical formulations for subcutaneous use, comprising either cagrilintide or semaglutide.

Thus far, it has not been considered possible to co-formulate semaglutide and cagrilintide, due to the different physicochemical properties of these active pharmaceutical ingredients. Semaglutide, a GLP-1 receptor agonist, has an isoelectric point that is incompatible with the optimal pH of cagrilintide, an amylin receptor agonist. Semaglutide is optimally stable at pH 7.4 and has previously needed to be formulated in a neutral to slightly basic solution of pH 7-8, to ensure its solubility in aqueous solution. Cagrilintide is optimally stable at pH 4.0 and has needed to be formulated in acidic solution, increasing pH accelerating the rate of its chemical degradation. The different physicochemical properties of cagrilintide and semaglutide preclude a simple mixture of these two peptides. The same applies to other GLP-1 receptor agonist and amylin receptor agonist combinations when the two have incompatible optimal pH ranges.

There remains a need in the art for a simple means of co-administering a GLP-1 receptor agonist such as semaglutide and an amylin receptor agonist such as cagrilintide.

### SUMMARY OF THE INVENTION

Disclosed herein is a means of co-formulating an amylin receptor agonist and a GLP-1 receptor agonist. Disclosed herein is a liquid pharmaceutical formulation comprising an amylin receptor agonist, a GLP-1 receptor agonist and a cyclodextrin comprising hydrophilic chemical substitutions such as hydroxypropyl substitutions. The cyclodextrin may be of the hydroxypropyl-substituted alpha type, comprising six ring-arranged glucose units. The cyclodextrin may be of the hydroxypropyl-substituted beta type, comprising seven ring-arranged glucose units. The pharmaceutical formulation may further comprise a buffer such as histidine, a tonicity agent such as sorbitol and/or a surfactant such as polysorbate 20 and/or 80; and have a pH of about 5.5-6.5, such as a pH of 5.6-6.0. The pharmaceutical formulation disclosed herein may be administered by parenteral injection, preferably subcutaneous injection.

The pharmaceutical formulation disclosed herein may be used for the medical treatment of subjects with: overweight or obesity, with or without one or more associated co-morbidities; diabetes, with or without one or more associated co-morbidities; one or more cardiovascular diseases; non-alcoholic steatohepatitis (NASH); and/or cognitive impairment, such as that caused by Alzheimer's disease. The pharmaceutical formulation disclosed herein may improve convenience, treatment compliance and ultimately improved clinical outcome in such patients.

### DESCRIPTION

The current invention is a liquid pharmaceutical formulation comprising an amylin receptor agonist, a GLP-1 receptor agonist and a cyclodextrin comprising hydroxypropyl substitutions.

The pharmaceutical formulation disclosed herein may comprise two active pharmaceutical ingredients, namely a GLP-1 receptor agonist and an amylin receptor agonist.

Disclosed herein is a means of co-formulating an amylin receptor agonist and a GLP-1 receptor agonist, wherein the GLP-1 receptor agonist has an isoelectric point that precludes its co-formulation in the pH range enabling chemical stability of the amylin receptor agonist. Disclosed herein is a means of co-formulating a GLP-1 receptor agonist having an isoelectric point (pI) of less than 6.5, preferably less than 6.0, such as 3.5-6.0, such as 3.0-5.0, such as 4.0-6.0, and an amylin receptor agonist.

The optimal pH of the amylin receptor agonist is the pH at which it is, chemically and physically, most stable. The person skilled in the art can easily find the amylin receptor agonist's optimal pH by testing its chemical and physical stability, in an aqueous solution essentially consisting of the amylin receptor agonist, a buffer and water for injection, across the pH range.

The optimal pH of the GLP-1 receptor agonist is the pH at which it is, chemically and physically, most stable. The person skilled in the art can easily find the GLP-1 receptor agonist's optimal pH by testing its chemical and physical stability, in an aqueous solution essentially consisting of the GLP-1 receptor agonist, a buffer and water for injection, across the pH range. The physical stability of the GLP-1 receptor agonist may be a reflection of its isoelectric point, which may coincide with the pH where poorest physical stability might be expected.

As will be apparent to the person skilled in the art, the chemical stability and purity of any GLP-1 receptor agonist and/or any amylin receptor agonist in any liquid formulation can be quantified by means of, e.g., reversed phase (ultra) high performance liquid chromatography (RP-UHPLC or RP-HPLC) and/or by measuring the percentage of high molecular weight protein (% HMWP) by means of, e.g., size exclusion chromatography (SEC).

As will be apparent to the person skilled in the art, the physical stability of a GLP-1 receptor agonist and/or any amylin receptor agonist in any liquid formulation can be quantified by measuring particle formation and/or fibrillation by means of micro-flow imaging (MFI) or a Thioflavin T (ThT) fluorescence stress assay, respectively.

Disclosed here is a means of formulating an amylin receptor agonist and a GLP-1 receptor agonist whose optimal pHs differ by at least about two pH units, such as 2-5 pH units, such as 2-4 pH units, such as 3-5 pH units.

The GLP-1 receptor agonist may be semaglutide. The amylin receptor agonist may be cagrilintide or a biologically active metabolite or degradation product of cagrilintide. The composition of the formulation disclosed herein preserves/improves the chemical and physical stability of the active pharmaceutical ingredients, even when co-formulated at pH 5.5-6.5, such as pH 5.6-6.0; preserves the pharmacokinetic profiles of the active pharmaceutical ingredients in terms of their bioavailability and exposure; and exhibits an acceptable local tolerance upon subcutaneous injection.

The terms “pharmaceutical formulation”, “co-formulation” and “drug product” may herein be used interchangeably to refer to a liquid pharmaceutical formulation comprising a GLP-1 receptor agonist and an amylin receptor agonist.

The pharmaceutical formulation disclosed herein is suitable for parenteral injection, preferably subcutaneous injection.

### Amylin

The term “amylin” herein refers to a polypeptide having the same amino acid sequence as an endogenous amylin, such as human amylin.

### Amylin Receptor

An amylin receptor agonist may bind to and activate the calcitonin receptor (CTR) and/or the amylin receptors (AMYRs). The latter consist of heterodimers of two components: the calcitonin receptor (CTR) and one of three receptor activity-modifying proteins (RAMP1-3) resulting in three possible complexes, AMYR1-3.

### Amylin Receptor Agonists

The pharmaceutical formulations disclosed herein comprise an amylin receptor agonist. An “amylin receptor agonist” may be defined as a chemical entity which is capable of binding to an amylin receptor and is capable of activating or “agonising” it. In the context of the current invention, the amylin receptor agonist is capable of binding to and activating at least the amylin receptor 3 (AMYR3). The amylin receptor agonist may also be capable of agonising the calcitonin receptor, the amylin receptor 1 (AMYR1) and/or the amylin receptor 2 (AMYR2).

Examples of endogenous amylin receptor agonists are human amylin and human calcitonin. Examples of exogenous amylin receptor agonists are cagrilintide and pramlintide (the active pharmaceutical ingredient in Symlin®).

The amylin receptor agonist is capable of activating the amylin receptor; in other words, it is “potent” on the amylin receptor. The in vitro potency of the amylin receptor agonist on amylin receptor 3 may be measured as described in WO/2022129526, Assay 2. The potency of the compound may be described by means of its EC₅₀ value, wherein EC₅₀ represents the concentration of compound upon which 50% of its maximal effect is observed. The lower the EC₅₀ value, the more potent the compound.

When tested as described as described in WO/2022129526, Assay 2, the amylin receptor agonist as disclosed herein may have an EC₅₀ value of less than 300 μM, such as less than 200 μM, such as less than 150 μM, preferably less than 100 μM, such as less than 75 μM, preferably less than 50 μM, such as less than 40 μM, such as less than 30 μM, such as less than 20 μM, such as less than 10 μM.

### Cagrilintide

The amylin receptor agonist in the pharmaceutical formulation disclosed herein may be cagrilintide or a biologically active metabolite or degradation product of cagrilintide.

Cagrilintide, also known as AM833, is the compound of Example 53 in WO2012/168432: N-alpha-[(S)-4-Carboxy-4-(19-carboxynonadecanoylamino)butyryl]-[Glu14,Arg17,Pro37]-pramlintide. Cagrilintide may be prepared as described in WO2012/168432, pages 153-155.

Cagrilintide may be in the form of a salt, preferably a pharmaceutically acceptable salt.

A biologically active metabolite or degradation product of cagrilintide may have an aspartate (Asp) in position 21 or 22. A biologically active metabolite or degradation product of cagrilintide may have an iso-aspartate (iso-Asp) in position 21 or 22.

When the potency of cagrilintide was tested using the procedure described in WO/2022129526, Assay 2, cagrilintide had an EC₅₀ value of about 11 μM (WO/2022/129526, Tables 4b and 4c).

The concentration of cagrilintide in the pharmaceutical formulation disclosed herein may be from about 0.25 mg/ml to about 22 mg/ml.

The pharmaceutical formulation disclosed herein may comprise cagrilintide in a concentration of about 0.33-18 mg/ml; such as 0.25-0.5 mg/ml, such as about 0.33 mg/ml; such as 0.5-1.0 mg/ml, such as about 0.67 mg/ml; such as 1.0-1.5 mg/ml, such as about 1.33 mg/ml; such as 1.5-2.0 mg/ml, such as about 1.5 mg/ml; such as 2.0-2.5 mg/ml; such as 2.5-3.0 mg/ml; such as 3.0-3.5 mg/ml; such as about 3.2 mg/ml; such as 3.5-4.0 mg/ml; such as 4.0-5.0 mg/ml; such as 5.0-6.0 mg/ml; such as 6.0-7.0 mg/ml, such as 7.0-8.0 mg/ml, such as 8.0-9.0 mg/ml, such as 9.0-10.0 mg/ml, such as about 9.6 mg/ml; such as 10-11 mg/ml, such as 11.0-12.0 mg/ml, such as 11-13 mg/ml; such as 13-22 mg/ml, such as about 18 mg/ml; such as about 20-22 mg/ml.

The pharmaceutical formulation disclosed herein may comprise no more than 22 mg/ml cagrilintide. The pharmaceutical formulation disclosed herein may comprise no more than 12 mg/ml cagrilintide.

### GLP-1

The term “GLP-1” or “native GLP-1” herein refers to human Glucagon-Like Peptide-1 (GLP-1 (7-37)).

### GLP-1 Receptor Agonist

The pharmaceutical formulations disclosed herein comprise a GLP-1 receptor agonist. A “GLP-1 receptor agonist” may be defined as a ligand which is capable of binding to the GLP-1 receptor and producing a biological response similar to that of the endogenous ligand, glucagon-like peptide 1 (GLP-1 (7-37)). A “full” GLP-1 receptor agonist may be defined as a GLP-1 receptor agonist which is capable of eliciting a biological response of the same magnitude as GLP-1 (7-37).

Examples of exogenous GLP-1 receptor agonists include semaglutide (the active pharmaceutical ingredient in Ozempic®, Rybelsus® and Wegovy®), liraglutide (the active pharmaceutical ingredient in Victoza® and Saxenda®), tirzepatide (the active pharmaceutical ingredient in Mounjaro®) and dulaglutide (the active pharmaceutical ingredient in Trulicity®).

The GLP-1 receptor agonist is capable of binding to and activating, or “agonising” the GLP-1 receptor; in other words, it is “potent” on the GLP-1 receptor. The in vitro potency of the GLP-1 receptor agonist on the GLP-1 receptor may be measured as described in WO/2022/129526, Assay 1. The potency of the compound may be described by means of its EC₅₀ values, wherein EC₅₀ represents the concentration of compound upon which 50% of its maximal effect is observed. The lower the EC₅₀ value, the more potent the compound.

When tested as described in WO/2022/129526, Assay 1, the GLP-1 receptor agonist disclosed herein may have an EC₅₀ value of less than 300 μM, such as less than 200 pM, such as less than 150 pM, preferably less than 100 pM, such as less than 75 pM, even more preferably less than 50 pM, such as less than 40 pM, such as less than 30 pM, such as less than 20 pM, such as less than 10 pM.

### Semaglutide

Semaglutide is a GLP-1 receptor agonist also known as N^{6.26}-{18-[N-(17-carboxyheptadecanoyl)-L-γ-glutamyl]-10-oxo-3,6, 12, 15-tetraoxa-9,18-diazaoctadecanoyl}-[8-(2-amino-2-propanoic acid),34-L-arginine] human glucagon-like peptide 1 (7-37). Semaglutide was described in WO2006/097537 and in J. Med. Chem. 2015, 58, 18, 7370-7380 and may be manufactured using methods well known to the person skilled in the art, such as that briefly described in WO2006/097537, Example 4.

Semaglutide may be present in the current pharmaceutical formulation in its fully or partly ionised form; for example one or more carboxylic acid groups (—COOH) may be deprotonated into the carboxylate group (—COO—) and/or one or more amino groups (—NH₂) may be protonated into the —NH₃⁺group.

Semaglutide may be in the form of a salt, preferably a pharmaceutically acceptable salt.

When the potency of semaglutide was tested according to the procedure described in WO/2022/129526, Assay 1, semaglutide had an EC₅₀ value of about 5.5 uM (see WO/2022/129526, Tables 4b and 4c).

The concentration of semaglutide in the pharmaceutical formulation disclosed herein may be from about 0.25 mg/ml to about 22 mg/ml.

The pharmaceutical formulation may comprise semaglutide in a concentration of about 0.33-18 mg/ml; such as 0.25-0.5 mg/ml, such as about 0.33 mg/ml; such as 0.5-1.0 mg/ml, such as about 0.67 mg/ml; such as 1.0-1.5 mg/ml, such as about 1.33 mg/ml; such as 1.5-2.0 mg/ml, such as about 1.5 mg/ml; such as 2.0-2.5 mg/ml; such as about 2.2 mg/ml, such as 2.5-3.0 mg/ml; such as 3.0-3.5 mg/ml; such as about 3.2 mg/ml; such as 3.5-4.0 mg/ml; such as 4.0-5.0 mg/ml; such as about 4.8 mg/ml; such as 5.0-6.0 mg/ml; such as 6.0-7.0 mg/ml, such as about 6.4 mg/ml; such as 7.0-8.0 mg/ml, such as about 8.0 mg/ml; such as 8.0-9.0 mg/ml, such as 9.0-10.0 mg/ml, such as about 9.6 mg/ml; such as 10-11 mg/ml, such as about 10.7 mg/ml; such as 11.0-12.0 mg/ml, such as 11-13 mg/ml; such as about 12.8 mg/ml; such as 13-22 mg/ml, such as about 16 mg/ml; such as about 18 mg/ml; such as about 20-22 mg/ml.

The pharmaceutical formulation disclosed herein may comprise no more than 22 mg/ml semaglutide. The pharmaceutical formulation disclosed herein may comprise no more than 12 mg/ml semaglutide.

### Methods of Manufacture

The GLP-1 receptor agonist and/or amylin receptor agonist in the pharmaceutical formulation disclosed herein may, for instance, be produced by classical peptide synthesis, e.g. solid phase peptide synthesis using t-Boc or Fmoc chemistry, or other well established techniques, see e.g. Greene and Wuts, “Protective Groups in Organic Synthesis”, John Wiley & Sons, 1999; Florencio Zaragoza Dorwald, “Organic Synthesis on Solid Phase”, Wiley-VCH Verlag GmbH, 2000; and “Fmoc Solid Phase Peptide Synthesis”, Edited by W.C. Chan and P.D. White, Oxford University Press, 2000.

Alternatively, the compounds may be produced by recombinant methods, e.g. by culturing a host cell containing a DNA sequence encoding the peptide sequence and capable of expressing the peptide, in a suitable nutrient medium under conditions permitting the expression of the peptide. Non-limiting examples of host cells suitable for expression of these peptides are Escherichia coli, Saccharomyces cerevisiae and mammalian BHK or CHO cell lines.

### Isoelectric Point

The isoelectric point (pI) of a molecule is the pH at which the molecule carries no net charge. The pl of a peptide may be theoretically calculated from the pK values of its amino acids and of the terminal amine and carboxyl groups and can be used to predict the solubility of the peptide at a given pH.

The theoretically calculated isoelectric point of the GLP-1 receptor agonist may be in the range of 3.5-6.5, such as 3.5-6.0, such as 4.0-6.0, such as 3.8-4.9, such as 4.0-4.5. Semaglutide has a theoretically calculated isoelectric point of about 4.37.

The theoretically calculated isoelectric point of the amylin receptor agonist may have an isoelectric point (pl) in the range of 8-12, such as 8-9. Cagrilintide has a theoretically calculated isoelectric point of about 8.56.

### Cyclodextrin

The pharmaceutical formulation disclosed herein comprises a cyclodextrin comprising hydroxypropyl substitutions.

The pharmaceutical formulation may comprise about 10-25% w/v of a cyclodextrin comprising hydroxypropyl substitutions. The pharmaceutical formulation may comprise more than 10% w/v of a cyclodextrin comprising hydroxypropyl substitutions. The pharmaceutical formulation may comprise less than 22% w/v of a cyclodextrin comprising hydroxypropyl substitutions. The pharmaceutical formulation may comprise about 10-20% w/v, about 15-25% w/v, about 12-18% w/v, about 10-17.5% w/v, about 11.25-15%, such as about 15% w/v of a cyclodextrin comprising hydroxypropyl substitutions.

Cyclodextrins are oligosaccharide starch derivatives consisting of 6, 7 or 8α-(1,4)-linked glucopyranose (glucose) units arranged cyclically and denoted the alpha, beta or gamma type, respectively. The cyclodextrins have a wide range of applications, amongst others as pharmaceutical excipients [P. Breen & S. S. Jambhekar, Cyclodextrins in pharmaceutical formulations II: solubilization, binding constant, and complexation efficiency, Drug Discovery Today, Volume 21, Number 2 Feb. 2016]. Guidelines on their use as pharmaceutical excipients have been described by the European Medicines Agency [Background review for cyclodextrins used as excipients, 2014, EMA/CHMP/333892/2013, Committee for Human Medicinal Products (CHMP)], [Cyclodextrins used as excipients, 2017, EMA/CHMP/333892/2013, Committee for Human Medicinal Products (CHMP)]. Cyclodextrin types that do not carry hydrophilic substitutions have poor solubility and are rarely used for parenteral drug products.

In order to improve the solubility of cyclodextrins, the hydroxyl groups of the glucose units of the cyclodextrins may be substituted by a varying number of hydrophilic chemical substitutions e.g. by hydroxypropyl groups, leading to differences in degree of substitution which can be described as either the average number of hydroxypropyl per cyclodextrin molecule (abbreviated DS) or the molar substitution degree corresponding to the average number of hydroxypropyl per glucose units present in the cyclodextrin in question (abbreviated MS). The value of hydroxypropyl per cyclodextrin can be achieved by multiplication of the molar substitution degree by the number of glucose units comprised in the cyclodextrin in question. Difference in degree of substitution can result in alterations in physicochemical properties such as surface activity and complexing abilities. The hydroxyl groups may also be chemically substituted by groups of sulfobutylether. These mostly hydrophilic modifications have yielded cyclodextrin derivates highly suitable for parenteral administration [Cyclodextrins used as excipients, 2017, EMA/CHMP/333892/2013, Committee for Human Medicinal Products (CHMP)]. Cyclodextrins comprising hydroxypropyl substitutions are commonly abbreviated HP-CDs whereas cyclodextrins comprising sulfobutylether substitutions are abbreviated SBE-CDs.

The cyclodextrins comprising hydrophilic substitutions adopt what may be described as cone-liked shapes having a hydrophobic inner cavity and a hydrophilic outer surface formed by the many hydrophilic substitutions capable of forming hydrogen bonds with neighbouring water molecules, thereby improving water solubility [T. Loftsson, Cyclodextrins in Parenteral Formulations, Journal of Pharmaceutical Sciences, 2020, 1-11].

Their hydrophobic microenvironment inside the cavity of these cone-like structures enables them to form drug-to-cyclodextrin complexes mainly through hydrophobic interactions [T. Loftsson, Cyclodextrins in Parenteral Formulations, Journal of Pharmaceutical Sciences, 2020, 1-11]. As a complex is formed between cyclodextrin and a drug molecule carrying one or more hydrophobic regions, these as well as the hydrophobic cavity of cyclodextrin become shielded from water, thereby increasing the solubility of the complex compared to the solubility of the individual constituents. Also, once the complex between cyclodextrin and peptide molecules is formed, it impairs the intermolecular interactions that typically leads to aggregation [T. Loftsson, Cyclodextrins in Parenteral Formulations, Journal of Pharmaceutical Sciences, 2020, 1-11].

The pharmaceutical formulation disclosed herein preferably comprises a cyclodextrin of the hydroxypropyl-substituted alpha type and/or a cyclodextrin of the hydroxypropyl-substituted beta type.

Unexpectedly, such cyclodextrin carrying hydroxypropyl substitutions was found superior, in its ability to stabilise a co-formulation of cagrilintide and semaglutide, than the same cyclodextrin type carrying sulfobutylether substitutions.

The pharmaceutical formulation disclosed herein may comprise a cyclodextrin of the hydroxypropyl-substituted alpha type, comprising six ring-arranged glucose units. The hydroxypropyl substituted cyclodextrin of the alpha type is abbreviated HP-A-CD. Hydroxypropyl-alpha-cyclodextrins (CAS: 128446-33-3/99241-24-4) are commercially available, with an average molar substitution (MS) of 0.8 and a molar substitution range of 0.5-0.9.

The pharmaceutical formulation disclosed herein may comprise hydroxypropyl-alpha-cyclodextrin having a minimum of about 0.4 hydroxypropyls per glucose unit. The pharmaceutical formulation disclosed herein may comprise hydroxypropyl-alpha-cyclodextrin having a maximum of about 1.0 hydroxypropyls per glucose unit.

The pharmaceutical formulation disclosed herein may comprise hydroxypropyl-alpha-cyclodextrin having a molar substitution range of 0.5-0.9 hydroxypropyls per glucose unit. The pharmaceutical formulation disclosed herein may comprise hydroxypropyl-alpha-cyclodextrin having an average molar substitution (MS) of about 0.8 hydroxypropyls per glucose unit.

The pharmaceutical formulation disclosed herein may comprise a cyclodextrin of the hydroxypropyl-substituted beta type, comprising seven ring-arranged glucose units. The hydroxypropyl substituted cyclodextrin of the beta type is abbreviated HP-B-CD.

Hydroxypropyl-beta-cyclodextrins are well known pharmaceutical excipients, typically used in small molecule pharmaceutical formulations, primarily to increase solubility and bioavailability [T. Loftsson, Cyclodextrins in Parenteral Formulations, Journal of Pharmaceutical Sciences, 2020, 1-11]. Thus far, the use of cyclodextrins and cyclodextrin substituted derivatives in protein and peptide-based pharmaceutical formulations is limited.

The commercially available hydroxypropyl substitution degrees (DS) for hydroxypropyl-beta-cyclodextrins as pharmaceutical excipients ranges between 2.8 and 10.5 according to the European and US pharmacopoeia [USP 38 NF 33, Pharm Eur 8, as estimated by methods described in USP <761>/Pharm. Eur. 2.2.33], corresponding to 0.4-1.5 hydroxypropyl per glucose unit (MS). Commercially available cyclodextrins such as hydroxypropyl-beta-cyclodextrins are usually described by means of the average molar substitutions (MS) of their molar substitution ranges.

Hydroxypropyl-beta-cyclodextrins (CAS: 128446-35-5/94035 Feb. 6) are commercially available for use as excipients, with average molar substitutions (MS) including: MS 0.62, with a molar substitution range of 0.58-0.68; MS 0.67, with a molar substitution range from (0.6-0.9); MS 0.68, with a molar substitution range from (0.58-0.72); MS 0.84, with a molar substitution range from (0.8-1.0); MS 0.92, with a molar substitution range from (0.81-0.99); MS 1.08, with a molar substitution range from (0.86-1.14); each value describing the number of hydroxypropyls per glucose unit.

The pharmaceutical formulation disclosed herein may comprise hydroxypropyl-beta-cyclodextrin having a minimum of about 0.4 hydroxypropyls per glucose unit. The pharmaceutical formulation disclosed herein may comprise hydroxypropyl-beta-cyclodextrin having a maximum of about 1.0 hydroxypropyls per glucose unit.

The pharmaceutical formulation disclosed herein may comprise hydroxypropyl-beta-cyclodextrin having a molar substitution range of 0.58-1.0 hydroxypropyls per glucose unit. The pharmaceutical formulation disclosed herein may comprise hydroxypropyl-beta-cyclodextrin having an average molar substitution (MS) range of about 0.62-0.92 hydroxypropyls per glucose unit.

The pharmaceutical formulation disclosed herein may comprise hydroxypropyl-beta-cyclodextrin having an average molar substitution (MS) of about 0.62-0.84 hydroxypropyls per glucose unit.

The pharmaceutical formulation disclosed herein may comprise hydroxypropyl-beta-cyclodextrin having about 0.4-0.75 hydroxypropyls per glucose unit.

The pharmaceutical formulation disclosed herein may comprise hydroxypropyl-beta-cyclodextrin having about 0.75 hydroxypropyls per glucose unit.

The pharmaceutical formulation disclosed herein may comprise hydroxypropyl-beta-cyclodextrin having an average molar substitution (MS) of about 0.62. The pharmaceutical formulation disclosed herein may comprise hydroxypropyl-beta-cyclodextrin having about 0.58-0.68 hydroxypropyls per glucose unit.

The pharmaceutical formulation disclosed herein may comprise hydroxypropyl-beta-cyclodextrin having an average molar substitution (MS) of about 0.68. The pharmaceutical formulation disclosed herein may comprise hydroxypropyl-beta-cyclodextrin having about 0.58-0.72 hydroxypropyls per glucose unit.

The pharmaceutical formulation disclosed herein may comprise hydroxypropyl-beta-cyclodextrin having an average molar substitution (MS) of about 0.67. The pharmaceutical formulation disclosed herein may comprise hydroxypropyl-beta-cyclodextrin having about 0.6-0.9 hydroxypropyls per glucose unit.

The pharmaceutical formulation disclosed herein may comprise hydroxypropyl-beta-cyclodextrin having an average molar substitution (MS) of about 0.84. The pharmaceutical formulation disclosed herein may comprise hydroxypropyl-beta-cyclodextrin having about 0.8-1.0 hydroxypropyls per glucose unit.

The pharmaceutical formulation disclosed herein may comprise hydroxypropyl-beta-cyclodextrin having an average molar substitution (MS) of about 0.92. The pharmaceutical formulation disclosed herein may comprise hydroxypropyl-beta-cyclodextrin having about 0.81-0.99 hydroxypropyls per glucose unit.

The pharmaceutical formulation disclosed herein may comprise 10-25% w/V, such as more than 10% w/v and less than 22% w/v, such as about 10-20% w/v, such as about 12-18% w/v, such as about 10-17.5% w/v, such as about 11.25-15%, such as about 15% w/v hydroxypropyl-beta-cyclodextrin having a minimum of about 0.4 hydroxypropyls per glucose unit and a maximum of about 1.0 hydroxypropyls per glucose unit; such as an average of 0.62-0.92 hydroxypropyls per glucose unit, such as about 0.75 hydroxypropyls per glucose unit; such as an average of 0.62-0.84 hydroxypropyls per glucose unit; such as about 0.4-0.75 hydroxypropyls per glucose unit; such as an average of 0.62 hydroxypropyls per glucose unit, such as about 0.58-0.68 hydroxypropyls per glucose unit.

### Further Excipients

The pharmaceutical formulation may comprise a buffer. The use of a buffer in pharmaceutical formulations is well-known to the skilled person. For convenience, reference is made to Remington: The Science and Practice of Pharmacy, 20th edition, 2000.

pH may be measured at “room temperature”, typically defined as 15-25° C. or 15-20° C. pH is preferably measured at about 20° C.

The pharmaceutical formulation disclosed herein may comprise a buffer having a pKa close to the desired pH of the solution. The pharmaceutical formulation may comprise a buffer having at least one pKa value of about 5.0-7.0. The pharmaceutical formulation may comprise a buffer having a pKa of about 5.0-7.0. The pharmaceutical formulation may comprise a buffer selected from the group consisting of histidine, citrate and/or phosphate. The buffer may be citrate, in a concentration of 3-30 mM. The buffer may be histidine, in a concentration of 3-30 mM. The buffer may be phosphate, in a concentration of 3-30 mM.

The pharmaceutical formulation may further comprise one or more agents for adjusting pH, such as NaOH and/or HCl.

The desired pH of the pharmaceutical formulation may be about 5.5-6.5. The pH is preferably 5.6-6.0. The pH may be about 5.6, such as about 5.7, such as about pH 5.8, such as about 5.9, such as about 6.0.

The pharmaceutical formulation may comprise a tonicity agent. The use of a tonicity agent in pharmaceutical formulations is well-known to the skilled person. For convenience, reference is made to Remington: The Science and Practice of Pharmacy, 20th edition, 2000.

The purpose of the tonicity agent is to protect living tissue when the formulation is injected into the body. The tonicity agent may be selected from the group consisting of mannitol, sorbitol or trehalose, or a combination thereof. In some embodiments, the tonicity agent is mannitol. In some embodiments, the tonicity agent is sorbitol. In some embodiments, the tonicity agent is trehalose.

The concentration of the tonicity agent is such as to render the formulation isotonic. Where the tonicity agent is mannitol, it may be present in a concentration of 16.5-37.5 mg/ml, such as about 20 mg/ml. Where the tonicity agent is sorbitol, it may be present in a concentration of about 10-40 mg/ml; such as about 16.5-37.5 mg/ml; such as about 10-30 mg/ml; such as about 16-28 mg/ml, such as about 16.5-25 mg/ml, such as about 16-26 mg/ml; such as about 16-24 mg/ml; such as about 26 mg/ml, such as about 24 mg/ml, such as about 22 mg/ml, such as about 20 mg/ml, such as about 18 mg/ml, such as about 16 mg/ml, such as about 12 mg/ml. Where the tonicity agent is trehalose, it may be present in a concentration of 33-75 mg/ml, such as about 38 mg/ml.

The pharmaceutical formulation may comprise a surfactant. The surfactant may further increase the physical stability and robustness of a formulation during its manufacture, storage and use as a medicament; for example, preserve the stability of a formulation when it is exposed to air inside a container. The use of surfactants in pharmaceutical formulations is well-known to the skilled person. For convenience, reference is made to Remington: The Science and Practice of Pharmacy, 20th edition, 2000.

The surfactant may be selected from the group consisting of polysorbate 20 and/or polysorbate 80. The surfactant may be polysorbate 20. The surfactant may be polysorbate 80.

The pharmaceutical formulation may comprise 0.01 mg/ml or more polysorbate 20 and up to 2.0, such as up to 1.5 mg/ml polysorbate 20, such as about 0.01-1.0 mg/ml polysorbate 20, such as about 0.05 mg/ml polysorbate 20.

The pharmaceutical formulation may comprise 0.01 mg/ml or more polysorbate 80 and up to 2.0, such as up to 1.5 mg/ml polysorbate 80, such as about 0.01-1.0 mg/ml polysorbate 80, such as about 0.05 mg/ml polysorbate 80.

The pharmaceutical formulation comprises water for injection (WFI). The pharmaceutical formulation may comprise more than 75% w/w water, such as 80% w/w water, such as about 85% w/w water, such as up to 90% w/w water.

The pharmaceutical formulation disclosed herein may comprise no preservative.

### Medical Utility

The pharmaceutical formulations disclosed herein may be for medical use.

The pharmaceutical formulation disclosed herein may be administered by parenteral injection. The pharmaceutical formulation disclosed herein may be administered by subcutaneous injection.

The term “treatment”, as used herein, refers to the medical therapy of any human or other vertebrate subject in need thereof. Said subject is expected to have undergone physical examination by a medical practitioner, or a veterinary medical practitioner, who has given a tentative or definitive diagnosis which would indicate that the use of said specific treatment is beneficial to the health of said human or other vertebrate. The timing and purpose of said treatment may vary from one individual to another, according to the status quo of the subject's health. Thus, said treatment may be prophylactic (preventative), palliative, symptomatic and/or curative.

The pharmaceutical formulation disclosed herein may be administered to a human subject.

The pharmaceutical formulation disclosed herein may be used in:
(i) the prevention and/or treatment of any form of diabetes and associated symptoms, such as hyperglycemia, type 2 diabetes, impaired glucose tolerance, type 1 diabetes, non-insulin dependent diabetes, maturity onset diabetes of the young (MODY), gestational diabetes and/or for the reduction of HbAlc;
(ii) the delaying or prevention of diabetic disease progression, such as progression in type 2 diabetes, delaying the progression of impaired glucose tolerance (IGT) to insulin-requiring type 2 diabetes and/or delaying the progression of non-insulin requiring type 2 diabetes to insulin-requiring type 2 diabetes;
(iii) the prevention and/or treatment of eating disorders, such as obesity, e.g. by decreasing food intake, suppressing appetite, inducing satiety, reducing body weight; treating or preventing binge eating disorder, food cravings, bulimia nervosa and/or obesity induced by the administration of an antipsychotic or a steroid; reducing gastric motility; and/or delaying gastric emptying;
(iv) the prevention and/or treatment of cardiovascular disease, such as the delaying or reduction of the development of a major adverse cardiovascular event (MACE) selected from the group consisting of cardiovascular death, non-fatal myocardial infarction, non-fatal stroke, revascularisation, hospitalisation for unstable angina pectoris, and hospitalisation for heart failure;
(v) the prevention and/or treatment of non-alcoholic fatty liver disease (NAFLD) and/or non-alcoholic steatohepatitis (NASH);
(vi) the prevention and/or treatment of cognitive disorders such as Alzheimer's disease.

In some embodiments, the indication is (i). In some embodiments the indication is (ii). In a still further particular aspect the indication is (iii). In a still further particular aspect, the indication is (iv). In a still further particular aspect, the indication is (v). In a still further particular aspect, the indication is (vi). In some embodiments, the indication is type 2 diabetes and/or obesity.

Generally, all subjects suffering from obesity are also considered to be suffering from overweight. Disclosed herein is a method for the treatment or prevention of obesity. Disclosed herein is use of the formulations disclosed herein for the treatment or prevention of obesity. In some embodiments the subject suffering from obesity is human, such as an adult human or a paediatric human (including infants, children, and adolescents).

Body mass index (BMI) is a measure of body fat based on height and weight. The formula for calculation is BMI=weight in kilograms/height in meters². A human subject suffering from obesity may have a BMI of 30 kg/m² or more; this subject may also be referred to as being obese. In some embodiments the human subject suffering from obesity may have a BMI of >35 or a BMI in the range of >30 to <40. In some embodiments the obesity is severe obesity or morbid obesity, wherein the human subject may have a BMI of ≥40.

Disclosed herein is a method for the treatment or prevention of overweight, optionally in the presence of at least one weight-related comorbidity. Disclosed herein is use of the formulations disclosed herein for the treatment or prevention of overweight, optionally in the presence of at least one weight-related comorbidity.

In some embodiments the subject suffering from overweight is human, such as an adult human or a paediatric human (including infants, children, and adolescents). In some embodiments a human subject suffering from overweight may have a BMI of 25 kg/m² or more, such as a BMI of 27 kg/m² or more. In some embodiments a human subject suffering from overweight has a BMI in the range of 25 to <30 or in the range of 27 to <30.

A raised BMI increases the risk of an individual developing any one of a wide range of diseases or co-morbidities. The weight-related comorbidity may be one, or a combination of, the diseases mentioned above. In some embodiments the weight-related comorbidity is selected from the group consisting of hypertension, diabetes (such as type 2 diabetes), dyslipidaemia, high cholesterol and obstructive sleep apnoea.

Disclosed herein is a method for reduction of body weight. A human to be subjected to reduction of body weight may have a BMI of 25 kg/m² or more, such as a BMI of 27 kg/m² or more (overweight) or a BMI of 30 kg/m² or more (obesity). In some embodiments the human to be subjected to reduction of body weight may have a BMI of 35 kg/m² or more or a BMI of 40 kg/m² or more. The term “reduction of body weight” may include treatment or prevention of obesity and/or overweight.

In some embodiments, administration of the semaglutide and cagrilintide pharmaceutical formulations disclosed herein may be used as an adjunct to a reduced-calorie diet and increased physical activity for chronic weight management in adult patients with an initial body mass index (BMI) of 30 kg/m² or more (obesity) or 27 kg/m² or more (overweight) in the presence of at least one weight-related comorbidity (e.g. hypertension, type 2 diabetes mellitus, or dyslipidaemia).

In some embodiments, administration of the semaglutide and cagrilintide pharmaceutical formulations disclosed herein may result in >15% weight loss, such as >20% weight loss, such as >25% weight loss, such as >30% weight loss, such as about 15-40% weight loss, such as about 20-35% weight loss, such as about 25-30% weight loss, within 26 weeks of the start of treatment.

In some embodiments, administration of the semaglutide and cagrilintide pharmaceutical formulations disclosed herein disclosed herein may result in >15% weight loss, such as >20% weight loss, such as >25% weight loss, such as >30% weight loss, such as about 15-40% weight loss, such as about 20-35% weight loss, such as about 25-30% weight loss, within 26 weeks of the start of treatment.

In some embodiments, administration of the semaglutide and cagrilintide pharmaceutical formulations disclosed herein results in a higher HbAic reduction, in %-points, compared to that which results from treatment with either semaglutide as sole active ingredient or cagrilintide as sole active ingredient.

### Dosages

The pharmaceutical formulation of the invention comprises a specific concentration of amylin receptor agonist and a specific concentration of GLP-1 receptor agonist. For example, as mentioned above, the pharmaceutical formulation may comprise from 0.25 to 22 mg/ml cagrilintide and from 0.25 to 22 mg/ml semaglutide. The doses of GLP receptor agonist and amylin receptor agonist administered in a single injection depend on the concentrations of these active ingredients within the pharmaceutical formulation and the volume of pharmaceutical formulation administered.

The pharmaceutical formulation of the invention may be administered as a single dose at predefined intervals.

A single dose of the pharmaceutical formulation disclosed herein may contain any one of the following doses of an amylin receptor agonist, such as cagrilintide, and a GLP-1 receptor agonist, such as semaglutide.

An effective amount of an amylin receptor agonist, such as cagrilintide, and a GLP-1 receptor agonist, such as semaglutide, may be administered to a subject in need thereof.

In some embodiments, the dose is administered approximately once weekly. In some embodiments, the interval between two fixed doses may be about 4 days, about 5 days, about 6 days, about 7 days, about 8 days, about 9 days or about 10 days. In a preferred embodiment, a fixed maintenance dose is administered approximately once every 7 days (once weekly).

In some embodiments, the dose is administered to an individual having any one or a combination of the diseases or co-morbidities listed above. In some preferred embodiments, the dose is administered a subject with obesity (body mass index [BMI] >30 kg/m²). In some preferred embodiments, the dose is administered to individuals that are overweight (BMI ≥27 kg/m²-<30 kg/m²) and that have at least one weight-related co-morbidity (e.g. hypertension, type 2 diabetes mellitus, or dyslipidaemia).

In some embodiments, once weekly treatment results in a statistically significant, dose-dependent, reduction in body weight.

In some preferred embodiments, the dose is administered as an adjunct to diet and exercise to improve glycemic control in adults with type 2 diabetes mellitus.

Upon initiation of treatment, it may be beneficial to administer ascending doses of an amylin receptor agonist, such as cagrilintide, and a GLP-1 receptor agonist, such as semaglutide, to individuals in need thereof. Once the individual is acclimatised to the treatment, it may be beneficial to administer maintenance doses of an amylin receptor agonist such as cagrilintide and a GLP-1 receptor agonist such as semaglutide to individuals in need thereof.

In some embodiments, treatment is once weekly and the dose-escalation period is 16 weeks.

In some embodiments, treatment is once weekly and dose escalation occurs approximately once weekly.

In some embodiments, treatment is once weekly and dose escalation occurs approximately once every other week.

In some embodiments, treatment is once weekly and dose escalation occurs approximately once every three weeks.

In some embodiments, treatment is once weekly and dose escalation occurs approximately once every four weeks.

The dose of amylin receptor agonist administered may be about 0.25-16 mg, such as about 0.25-9.0 mg, such as about 0.25-4.5 mg, such as about 0.25-2.4 mg.

The dose of cagrilintide administered may be about 0.25-16 mg, such as about 0.25-9.0 mg, such as about 0.25-4.5 mg, such as about 0.25-2.4 mg.

In some embodiments, the dose of cagrilintide administered is about 0.25 mg.

In some embodiments, the dose of cagrilintide administered is about 0.5 mg.

In some embodiments, the dose of cagrilintide administered is about 1.0 mg.

In some embodiments, the dose of cagrilintide administered is about 1.5 mg.

In some embodiments, the dose of cagrilintide administered is about 1.7 mg.

In some embodiments, the dose of cagrilintide administered is about 2.4 mg.

In some embodiments, the dose of cagrilintide administered is about 3.4 mg.

In some embodiments, the dose of cagrilintide administered is about 3.6 mg.

In some embodiments, the dose of cagrilintide administered is about 4.5 mg.

In some embodiments, the dose of cagrilintide administered is about 7.2 mg.

In some embodiments, the dose of cagrilintide administered is about 8.0 mg.

In some embodiments, the dose of cagrilintide administered is about 9.0 mg.

In some embodiments, the dose of cagrilintide administered is about 16.0 mg.

The dose of GLP-1 receptor agonist administered may be about 0.25-16 mg, such as about 0.25-9.0 mg, such as about 0.25-4.5 mg, such as about 0.25-2.4 mg.

The dose of semaglutide administered may be about 0.25-16 mg, such as about 0.25-9.0 mg, such as about 0.25-4.5 mg, such as about 0.25-2.4 mg.

In some embodiments, the dose of semaglutide administered is about 0.25 mg.

In some embodiments, the dose of semaglutide administered is about 0.5 mg.

In some embodiments, the dose of semaglutide administered is about 1.0 mg.

In some embodiments, the dose of semaglutide administered is about 1.5 mg.

In some embodiments, the dose of semaglutide administered is about 1.7 mg.

In some embodiments, the dose of semaglutide administered is about 2.4 mg.

In some embodiments, the dose of semaglutide administered is about 3.6 mg.

In some embodiments, the dose of semaglutide administered is about 4.5 mg.

In some embodiments, the dose of semaglutide administered is about 4.8 mg.

In some embodiments, the dose of semaglutide administered is about 6.0 mg.

In some embodiments, the dose of semaglutide administered is about 6.9 mg.

In some embodiments, the dose of semaglutide administered is about 7.2 mg.

In some embodiments, the dose of semaglutide administered is about 8.0 mg.

In some embodiments, the dose of semaglutide administered is about 9.0 mg.

In some embodiments, the dose of semaglutide administered is about 12 mg.

In some embodiments, the dose of semaglutide administered is about 16.0 mg. In some embodiments, the dose of semaglutide administered is about 16.0 mg.

In some embodiments, the ratio of amylin receptor agonist to GLP-1 receptor agonist is about 1:2. In some embodiments, the ratio of cagrilintide to semaglutide is about 1:2.

In some embodiments, the dose of cagrilintide is about 0.125 mg and the dose of semaglutide is about 0.25 mg.

In some embodiments, the dose of cagrilintide is about 0.25 mg and the dose of semaglutide is about 0.5 mg.

In some embodiments, the dose of cagrilintide is about 0.5 mg and the dose of semaglutide is about 1.0 mg.

In some embodiments, the dose of cagrilintide is about 0.75 mg and the dose of semaglutide is about 1.5 mg.

In some embodiments, the dose of cagrilintide is about 0.85 mg and the dose of semaglutide is about 1.7 mg.

In some embodiments, the dose of cagrilintide is about 1.2 mg and the dose of semaglutide is about 2.4 mg.

In some embodiments, the dose of cagrilintide is about 2.25 mg and the dose of semaglutide is about 4.5 mg.

In some embodiments, the dose of cagrilintide administered is about 3.6 mg and the dose of semaglutide is about 7.2 mg.

In some embodiments, the dose of cagrilintide is about 4.0 mg and the dose of semaglutide is about 8.0 mg.

In some embodiments, the dose of cagrilintide is about 7.2 mg and the dose of semaglutide is about 14.4 mg.

In some embodiments, the dose of cagrilintide is about 8.0 mg and the dose of semaglutide is about 16.0 mg.

In some embodiments, the maintenance dose of cagrilintide is about 1.2 mg and the maintenance dose of semaglutide is about 2.4 mg.

In some embodiments, the maintenance dose of cagrilintide is about 2.25 mg and the dose of semaglutide is about 4.5 mg.

In some embodiments, the maintenance dose of cagrilintide is about 4.0 mg and the maintenance dose of semaglutide is about 8.0 mg.

In some embodiments, the maintenance dose of cagrilintide is about 8.0 mg and the maintenance dose of semaglutide is about 16.0 mg.

In some embodiments, the ratio of amylin receptor agonist to GLP-1 receptor agonist is about 1:1. In some embodiments, the ratio of cagrilintide to semaglutide is about 1:1.

In some embodiments, the dose of cagrilintide is about 0.25 mg and the dose of semaglutide is about 0.25 mg.

In some embodiments, the dose of cagrilintide is about 0.5 mg and the dose of semaglutide is about 0.5 mg.

In some embodiments, the dose of cagrilintide is about 1.0 mg and the dose of semaglutide is about 1.0 mg.

In some embodiments, the dose of cagrilintide is about 1.7 mg and the dose of semaglutide is about 1.7 mg.

In some embodiments, the dose of cagrilintide is about 2.4 mg and the dose of semaglutide is about 2.4 mg.

In some embodiments, the maintenance dose of cagrilintide is about 2.4 mg and the maintenance dose of semaglutide is about 2.4 mg.

In some embodiments, the dose of cagrilintide is about 4.5 mg and the dose of semaglutide is about 4.5 mg.

In some embodiments, the dose of cagrilintide is about 8.0 mg and the dose of semaglutide is about 8.0 mg.

In some embodiments, the dose of cagrilintide is about 16.0 mg and the dose of semaglutide is about 16.0 mg.

In some embodiments, the ratio of amylin receptor agonist to GLP-1 receptor agonist is between 1:1 and 1:7.

In some embodiments, the dose of cagrilintide is about 2.4 mg and the dose of semaglutide is about 2.4 mg to 16.0 mg.

In some embodiments, the dose of cagrilintide is about 2.4 mg and the dose of semaglutide is about 3.6 mg to 16.0 mg.

In some embodiments, the dose of cagrilintide is about 2.4 mg and the dose of semaglutide is about 2.4 mg to 13.5 mg.

In some embodiments, the dose of cagrilintide is about 2.4 mg and the dose of semaglutide is about 3.6 mg to 13.5 mg.

In some embodiments, the dose of cagrilintide is about 2.4 mg and the dose of semaglutide is about 3.6 mg.

In some embodiments, the dose of cagrilintide is about 2.4 mg and the dose of semaglutide is about 4.8 mg.

In some embodiments, the dose of cagrilintide is about 2.4 mg and the dose of semaglutide is about 6.0 mg.

In some embodiments, the dose of cagrilintide is about 2.4 mg and the dose of semaglutide is about 6.9 mg.

In some embodiments, the dose of cagrilintide is about 2.4 mg and the dose of semaglutide is about 7.2 mg.

In some embodiments, the dose of cagrilintide is about 2.4 mg and the dose of semaglutide is about 8.0 mg.

In some embodiments, the dose of cagrilintide is about 2.4 mg and the dose of semaglutide is about 12 mg.

In some embodiments, the dose of cagrilintide is about 3.4 mg and the dose of semaglutide is about 13.5 mg.

In some embodiments, cagrilintide and semaglutide are administered once-weekly at an initial dose of 0.25 mg and then escalated to the subsequent dosing levels of 0.5 mg, 1.0 mg and 1.7 mg until reaching the target/maintenance dose of 2.4 mg once-weekly.

In some embodiments, 0.25 mg cagrilintide and semaglutide are administered once-weekly and escalated every four weeks to the subsequent dosing levels of 0.5 mg, 1.0 mg and 1.7 mg, until reaching the target/maintenance dose of 2.4 mg once-weekly.

In some embodiments, 0.25 mg cagrilintide and semaglutide are administered once-weekly and escalated every four weeks to the subsequent dosing levels of 0.5 mg, 1.0 mg and 1.7 mg, until reaching the target/maintenance dose of 2.4 mg once-weekly.

In some embodiments, 0.25 mg cagrilintide and 0.25 mg semaglutide are administered once a week for four weeks (weeks 0-3) and escalated every four weeks to the subsequent dosing levels of 0.5 mg cagrilintide and 0.5 semaglutide (weeks 4-7), 1.0 mg cagrilintide and 1.0 semaglutide (weeks 8-11) and 1.7 mg cagrilintide and 1.7 mg semaglutide (weeks 12-15), until reaching the target/maintenance dose of 2.4 mg cagrilintide and 2.4 mg semaglutide mg once-weekly (weeks 16 and thereafter).

In some embodiments, cagrilintide and semaglutide are administered once-weekly at initial doses of 0.25 mg and then escalated to the subsequent dosing levels of 0.5 mg, 1.0 mg, 1.7 mg and 2.4 mg, until reaching the target/maintenance dose of 4.5 mg once-weekly.

In some embodiments, cagrilintide and semaglutide are administered once-weekly at initial doses of 0.25 mg and then escalated to the subsequent dosing levels of 0.5 mg, 1.0 mg, 1.7 mg, 2.4 mg, 3.6 mg and 4.5 mg, until reaching the target/maintenance dose of 7.2 mg once-weekly.

In some embodiments, cagrilintide and semaglutide are administered once-weekly at initial doses of 0.25 mg and then escalated to the subsequent dosing levels of 0.5 mg, 1.0 mg, 1.7 mg, 2.4 mg, 3.6 mg, 4.5 mg and 7.2 mg, until reaching the target/maintenance dose of 8.0 mg once-weekly.

In some embodiments, cagrilintide and semaglutide are administered once-weekly at initial doses of 0.25 mg and then escalated to the subsequent dosing levels of 0.5 mg, 1.0 mg, 1.7 mg, 2.4 mg, 3.6 mg, 4.5 mg, 7.2 mg and 8.0, until reaching the target/maintenance dose of 16.0 mg once-weekly.

Herein, specific values given in relation to numbers or intervals may be construed as being the specific value or as being the approximate value (such as plus or minus 10, 15 or 20 percent of the specific value, when amounts can be provided by weight; such as plus or minus 0.4, when pH is measured).

Following is a non-limiting list of embodiments of the present invention.

### EMBODIMENTS

1. A liquid pharmaceutical formulation comprising an amylin receptor agonist, a GLP-1 receptor agonist and a cyclodextrin comprising hydroxypropyl substitutions.
2. The liquid pharmaceutical formulation according to embodiment 1, wherein the GLP-1 receptor agonist has an isoelectric point that is incompatible with the optimal pH of the amylin receptor agonist.
3. The liquid pharmaceutical formulation according to any one of the preceding embodiments, wherein the optimal pH of the GLP-1 receptor agonist and the amylin receptor agonist differs by at least about two pH units, such as 2-5 pH units, such as 2-4 pH units, such as 3-5 pH units.
4. The liquid pharmaceutical formulation according to any of the preceding embodiments, wherein the optimal pH of the amylin receptor agonist is 3.5-4.5, such as about 4.0.
5. The pharmaceutical formulation according to any one of the preceding embodiments, wherein said amylin receptor agonist is cagrilintide.
6. The liquid pharmaceutical formulation according to any of the preceding embodiments, wherein said GLP-1 receptor agonist has an isoelectric point of less than 6.5, such as less than 6.0, such as 3.5-6.0, such as 3.0-5.0, such as 3.8-4.9.
7. The pharmaceutical formulation according to any one of the preceding embodiments, wherein said GLP-1 receptor agonist is semaglutide.
8. The pharmaceutical formulation according to any one of the preceding embodiments, wherein said cyclodextrin is of the hydroxypropyl-substituted alpha type comprising six ring-arranged glucose units and/or the hydroxypropyl-substituted beta type comprising seven ring-arranged glucose units.
9. The pharmaceutical formulation according to any one of the preceding embodiments, wherein said cyclodextrin is of the hydroxypropyl-substituted alpha type comprising six ring-arranged glucose units.
10. The pharmaceutical formulation according to any one of the preceding embodiments, wherein said cyclodextrin is of the hydroxypropyl-substituted beta type comprising seven ring-arranged glucose units.
11. The pharmaceutical formulation according to any one of the preceding embodiments, wherein said cyclodextrin comprises a maximum of about 1.0 hydroxypropyls per glucose unit.
12. The pharmaceutical formulation according to any one of the preceding embodiments, wherein said cyclodextrin comprises a maximum of about 0.92 hydroxypropyls per glucose unit.
13. The pharmaceutical formulation according to any one of the preceding embodiments, wherein said cyclodextrin comprises a maximum of about 0.75 hydroxypropyls per glucose unit.
14. The pharmaceutical formulation according to any one of the preceding embodiments, wherein said cyclodextrin comprises a maximum of about 0.68 hydroxypropyls per glucose unit.
15. The pharmaceutical formulation according to any one of the preceding embodiments, wherein said cyclodextrin comprises a minimum of about 0.4 hydroxypropyls per glucose unit.
16. The pharmaceutical formulation according to any one of the preceding embodiments, wherein said cyclodextrin comprises a minimum of about 0.58 hydroxypropyls per glucose unit.
17. The pharmaceutical formulation according to any one of the preceding embodiments, wherein said cyclodextrin comprises about 0.58-1.0 hydroxypropyls per glucose unit.
18. The pharmaceutical formulation according to embodiment 17, wherein said cyclodextrin comprises an average (MS) of 0.62-0.92 hydroxypropyls per glucose unit.
19. The pharmaceutical formulation according to embodiment 17, wherein said cyclodextrin comprises an average (MS) of about 0.62-0.84 hydroxypropyls per glucose unit.
20. The pharmaceutical formulation according to embodiment 17, wherein said cyclodextrin comprises an average (MS) of about 0.62 hydroxypropyls per glucose unit.
21. The pharmaceutical formulation according to embodiment 17, wherein said cyclodextrin comprises about 0.4-0.75 hydroxypropyls per glucose unit, such as about 0.58-0.68 hydroxypropyls per glucose unit.
22. The pharmaceutical formulation according to any one of the preceding embodiments, comprising about 10-25% w/v cyclodextrin.
23. The pharmaceutical formulation according to any one of the preceding embodiments, comprising more than 10% w/v cyclodextrin.
24. The pharmaceutical formulation according to any one of the preceding embodiments, comprising less than 22% w/v cyclodextrin.
25. The pharmaceutical formulation according to any one of the preceding embodiments, comprising less than 20% w/v cyclodextrin.
26. The pharmaceutical formulation according to any one of the preceding embodiments, comprising about 10-20% w/v of said cyclodextrin.
27. The pharmaceutical formulation according to any one of the preceding embodiments, comprising about 10-17.5% w/v cyclodextrin.
28. The pharmaceutical formulation according to any one of the preceding embodiments, comprising about 12-18% w/v cyclodextrin.
29. The pharmaceutical formulation according to any one of the preceding embodiments, comprising about 11.25-15% w/v cyclodextrin.
30. The pharmaceutical formulation according to any one of the preceding embodiments, comprising about 15% w/v cyclodextrin.
31. The pharmaceutical formulation according to any one of the preceding embodiments, comprising at least about 1 mg/ml of said GLP-1 receptor agonist.
32. The pharmaceutical formulation according to any one of the preceding embodiments, comprising a maximum of about 22 mg/ml of said GLP-1 receptor agonist.
33. The pharmaceutical formulation according to any one of the preceding embodiments, comprising about 1-12 mg/ml GLP-1 receptor agonist.
34. The pharmaceutical formulation according to any one of the preceding embodiments, comprising at least about 1 mg/ml of said amylin receptor agonist.
35. The pharmaceutical formulation according to any one of the preceding embodiments, comprising a maximum of about 22 mg/ml amylin receptor agonist.
36. The pharmaceutical formulation according to any one of the preceding embodiments, comprising about 1-12 mg/ml amylin receptor agonist.
37. The pharmaceutical formulation according to any one of the preceding embodiments, comprising 0.25-22 mg/ml cagrilintide.
38. The pharmaceutical formulation according to any one of the preceding embodiments, comprising 0.25-22 mg/ml semaglutide.
39. The pharmaceutical formulation according to any one of the preceding embodiments, comprising 0.25-22 mg/ml cagrilintide and 0.25-22 mg/ml semaglutide.
40. The pharmaceutical formulation according to any one of the preceding embodiments, comprising an effective amount of cagrilintide and semaglutide.
41. The pharmaceutical formulation according to any one of the preceding embodiments, further comprising a tonicity agent; with the proviso that the tonicity agent is not sodium chloride.
42. The pharmaceutical formulation according to the previous embodiment, wherein said tonicity agent is mannitol, sorbitol or trehalose, or a combination thereof.
43. The pharmaceutical formulation according to the previous embodiment, wherein said tonicity agent is mannitol.
44. The pharmaceutical formulation according to the preceding embodiment, comprising mannitol in a concentration of about 16.5-37.5 mg/ml, such as about 20 mg/ml.
45. The pharmaceutical formulation according to embodiment 41, wherein said tonicity agent is sorbitol.
46. The pharmaceutical formulation according to the preceding embodiment, comprising sorbitol in a concentration of about 10-40 mg/ml, such as about 10-30 mg/ml, such as about 16-28 mg/ml, such as about 16.5-37.5 mg/ml, such as about 16.5-25 mg/ml, such as about 16-24 mg/ml, such as about 24 mg/ml, such as about 20 mg/ml, such as about 16 mg/ml, such as about 12 mg/ml.
47. The pharmaceutical formulation according to embodiment 41, wherein said tonicity agent is trehalose.
48. The pharmaceutical formulation according to the preceding embodiment, comprising trehalose in a concentration of about 33-75 mg/ml, such as about 33-45 mg/ml, such as about 38 mg/ml.
49. The pharmaceutical formulation according to any one of the preceding embodiments, further comprising a buffer having at least one pKa of about 5.0-7.0.
50. The pharmaceutical formulation according to any one of the preceding embodiments, further comprising a buffer selected from the group consisting of histidine, citrate and/or phosphate.
51. The pharmaceutical formulation according to any one of the preceding embodiments, comprising a maximum of 30 mM buffer.
52. The pharmaceutical formulation according to any one of the preceding embodiments, comprising about 3-30 mM citrate.
53. The pharmaceutical formulation according to any one of the preceding embodiments, comprising about 3-30 mM histidine, such as 3-15 mM histidine, such as 3-10 mM histidine, such as about 6 mM histidine.
54. The pharmaceutical formulation according to any one of the preceding embodiments, comprising about 3-30 mM phosphate.
55. The pharmaceutical formulation according to any one of the preceding embodiments, further comprising a surfactant.
56. The pharmaceutical formulation according to the preceding embodiment, wherein said surfactant is polysorbate 20 and/or polysorbate 80.
57. The pharmaceutical formulation according to the preceding embodiment, comprising a maximum of about 2.0 mg/ml polysorbate 20 and/or polysorbate 80.
58. The pharmaceutical formulation according to the preceding embodiment, comprising a maximum of about 1.5 mg/ml polysorbate 20 and/or polysorbate 80.
59. The pharmaceutical formulation according to the preceding embodiment, wherein said surfactant is polysorbate 80.
60. The pharmaceutical formulation according to any one of the preceding embodiments, wherein the pH is about 5.5-6.5, preferably 5.6-6.0, such as about 5.7, such as about pH 5.8, such as about 5.9, such as about 6.0.
61. The pharmaceutical formulation according to any one of the preceding embodiments, comprising at least 75% w/w water, such as about 80% w/w water, such as about 85% w/w water, such as up to about 90% w/w water.
62. The pharmaceutical formulation according to any one of the preceding embodiments, essentially consisting of: an effective amount of cagrilintide and semaglutide, a cyclodextrin of the hydroxypropyl-substituted alpha and/or beta type comprising a minimum of about 0.4 hydroxypropyls per glucose unit and a maximum of about 1.0 hydroxypropyls per glucose unit, histidine, sorbitol, polysorbate 20 and/or 80 and about 75-90% w/w water; and having a pH of 5.6-6.0.
63. The pharmaceutical formulation according to any one of the preceding embodiments, essentially consisting of: an effective amount of cagrilintide and semaglutide; a cyclodextrin of the hydroxypropyl-substituted alpha and/or beta type, comprising 0.58-1.0 hydroxypropyls per glucose unit, histidine, sorbitol, polysorbate 20 and/or 80 and about 75-90% w/w water; and having a pH of 5.6-6.0.
64. The pharmaceutical formulation according to any one of the preceding embodiments, essentially consisting of: an effective amount of cagrilintide and semaglutide; a cyclodextrin of the hydroxypropyl-substituted alpha and/or beta type, comprising an average of 0.62-0.92 hydroxypropyls per glucose unit; histidine, sorbitol, polysorbate 20 and/or 80 and about 75-90% w/w water; and having a pH of 5.6-6.0.
65. The pharmaceutical formulation according to any one of the preceding embodiments, essentially consisting of: an effective amount of cagrilintide and semaglutide; a cyclodextrin of the hydroxypropyl-substituted alpha and/or beta type, comprising an average of 0.62-0.84 hydroxypropyls per glucose unit; histidine and/or citrate, sorbitol, polysorbate 20 and/or 80 and about 75-90% w/w water; and having a pH of 5.6-6.0.
66. The pharmaceutical formulation according to any one of the preceding embodiments, essentially consisting of: an effective amount of cagrilintide and semaglutide; a cyclodextrin of the hydroxypropyl-substituted alpha and/or beta type, comprising an average of 0.62-0.68 hydroxypropyls per glucose unit; histidine and/or citrate, sorbitol, polysorbate 20 and/or 80 and about 75-90% w/w water; and having a pH of 5.6-6.0.
67. The pharmaceutical formulation according to any one of the preceding embodiments, essentially consisting of: an effective amount of cagrilintide and semaglutide; a cyclodextrin of the hydroxypropyl-substituted alpha and/or beta type, comprising an average of 0.62 hydroxypropyls per glucose unit; histidine and/or citrate, sorbitol, polysorbate 20 and/or 80 and about 75-90% w/w water; and having a pH of 5.6-6.0.
68. The pharmaceutical formulation according to any one of the preceding embodiments, essentially consisting of: an effective amount of cagrilintide and semaglutide, a hydroxypropyl beta cyclodextrin comprising a maximum of about 0.75 hydroxypropyls per glucose unit, such as about 0.4-0.75 hydroxypropyls per glucose unit, histidine, sorbitol, polysorbate 80 and about 75-90% w/w water; and having a pH of 5.5-6.5.
69. The pharmaceutical formulation according to any one of the preceding embodiments, essentially consisting of: an effective amount of cagrilintide and semaglutide, a hydroxypropyl beta cyclodextrin comprising a maximum of about 0.75 hydroxypropyls per glucose unit, such as about 0.4-0.75 hydroxypropyls per glucose unit, histidine and/or citrate, sorbitol, polysorbate 20 and/or 80 and about 75-90% w/w water; and having a pH of 5.6-6.0.
70. The pharmaceutical formulation according to any one of the preceding embodiments, which essentially consists of:
an effective amount of cagrilintide and semaglutide,
more than 10% w/v and less than 22% w/v, such as 10-20% w/v cyclodextrin of the hydroxypropyl-substituted alpha and/or beta type (0.58-1.0 hydroxypropyls per glucose unit),
about 3-30 mM histidine,
about 10-40 mg/ml sorbitol,
up to 2.0 mg/ml polysorbate 20 and/or 80,
pH 5.6-6.0, preferably pH 5.8,
water for injection.
71. The pharmaceutical formulation according to any one of the preceding embodiments, which essentially consists of:
an effective amount of cagrilintide and semaglutide,
more than 10% w/v and less than 22% w/v, such as 10-20% w/v cyclodextrin of the hydroxypropyl-substituted alpha and/or beta type, comprising an average of 0.62-0.84 hydroxypropyls per glucose unit,
about 3-30 mM histidine and/or citrate,
about 10-40 mg/ml sorbitol,
up to 2.0 mg/ml polysorbate 20 and/or polysorbate 80,
pH 5.6-6.0, preferably pH 5.8,
water for injection.
72. The pharmaceutical formulation according to any one of the preceding embodiments, which essentially consists of:
0.25-22 mg/ml cagrilintide,
0.25-22 mg/ml semaglutide,
more than 10% w/v and less than 22% w/v, such as 10-20% w/v cyclodextrin of the hydroxypropyl-substituted alpha and/or beta type (0.58-1.0 hydroxypropyls per glucose unit),
about 6 mM histidine,
about 10-40 mg/ml sorbitol,
up to 2.0 mg/ml Polysorbate 20 and/or 80,
pH 5.6-6.0, preferably pH 5.8,
water for injection.
73. The pharmaceutical formulation according to any one of the preceding embodiments for use as a medicament.
74. The pharmaceutical formulation according to any one of embodiments 1-72 for use in the treatment of subjects with an initial body mass index (BMI) of 27 or more, such as 30 or more.
75. The pharmaceutical formulation according to any one of embodiments 1-72, for use in the treatment of subjects with an initial body mass index (BMI) of 27 or more and at least one weight-related co-morbidity.
76. The pharmaceutical formulation according to any one of embodiments 1-72, for use as an adjunct to a reduced-calorie diet and increased physical activity for chronic weight management in adult subjects with an initial body mass index (BMI) of 30 kg/m² or greater (obesity) or 27 kg/m² or greater (overweight) in the presence of at least one weight-related co-morbidity.
77. Use according to any one of embodiments 73-76, wherein said co-morbidity is diabetes and/or a cardiovascular disease and/or NASH.
78. The pharmaceutical formulation according to any one of embodiments 1-72, for use in the treatment of subjects with diabetes, such as type II diabetes.
79. The pharmaceutical formulation according to any one of embodiments 1-72, for use as an adjunct to diet and exercise to improve glycemic control in adults with type 2 diabetes mellitus.
80. The pharmaceutical formulation according to any one of embodiments 1-72, for use in the treatment and/or prevention of cardiovascular diseases.
81. The pharmaceutical formulation according to any one of embodiments 1-72, for use in the treatment and/or prevention of NASH.
82. The pharmaceutical formulation according to any one of embodiments 1-72, for use in the treatment and/or prevention of cognitive impairment, such as that caused by Alzheimer's disease.
83. The pharmaceutical formulation according to any one of embodiments 1-72 for use according to any one of embodiments 73-82, characterised in that the formulation is administered by parenteral injection.
84. The pharmaceutical formulation according to any one of embodiments 1-72 for use according to any one of embodiments 73-82, characterised in that the formulation is administered by subcutaneous injection.
85. The pharmaceutical formulation according to any one of embodiments 1-72 for use according to any one of embodiments 73-84, characterised in that the formulation is administered approximately once a week.
86. The pharmaceutical formulation according to any one of embodiments 1-72 for use according to any one of embodiments 73-85, wherein the ratio of the dose of cagrilintide administered to the dose of semaglutide administered is about 1:1.
87. The pharmaceutical formulation according to any one of embodiments 1-72 for use according to any one of embodiments 73-85, wherein the ratio of the dose of cagrilintide administered to the dose of semaglutide administered is from 1:1 to 1:7.
88. The pharmaceutical formulation according to any one of embodiments 1-72 for use according to any one of embodiments 73-85, wherein the ratio of the dose of cagrilintide administered to the dose of semaglutide administered is about 1:2.

### Examples

### Example 1: Effect of Hydroxypropyl-Beta-Cyclodextrin (Hp-β-Cd) on the Chemical Stability of Cagrilintide

This example demonstrated the ability of HP-B-CD to chemically stabilise cagrilintide, chemical stability being measured in terms of cagrilintide purity and cagrilintide-related high molecular weight protein (HMWP).

Cagrilintide is optimally stable at pH 4.0, the rate of its chemical degradation typically accelerating with an increase in pH. Surprisingly, a stable cagrilintide formulation was obtained at pH 6 when it was formulated with HP-B-CD.

### Composition

The compositions of cagrilintide formulations 1, 2 and 3 are shown in table 1.

TABLE 1
Composition of cagrilintide formulations 1, 2 and 3

| | Cagrilintide formulation | | |
|---|---|---|---|
| Ingredient | 1 | 2 | 3 |
| Cagrilintide drug | 18 | 18 | 18 |
| substance (mg/ml) | | | |
| HP-B-CD | 0 | 0 | 25 |
| KLEPTOSE ® | | | |
| (Roquette) (% w/v) | | | |
| (Average MS: 0.92, | | | |
| MS range: 0.81-0.99)¹ | | | |
| Citrate, 1 H₂O ² (mM) | 6.1 | 3.7 | 3.7 |
| Disodium hydrogen | 7.7 | 12.6 | 12.6 |
| phosphate, | | | |
| 2 H₂O ² (mM) | | | |
| HCl | q.s. | q.s. | q.s. |
| NaOH | q.s. | q.s. | q.s. |
| Water for | To make 1 ml | To make 1 ml | To make 1 ml |
| injection (WFI) | | | |
| pH | 4.0 | 6.0 | 6.0 |

¹MS: molar substitution, corresponds to hydroxypropyls per glucose unit
² Different buffer concentrations across formulations are used to ensure buffering at different pH

### Preparation Process

Each cagrilintide formulation was prepared by first dissolving the excipients in water and then dissolving cagrilintide drug substance in the excipient solution. The solution was pH adjusted and water was added to reach the final desired volume before being sterilised by filtration through a 0.22 μm sterile filter. After filtration, the formulation was filled in a 1 ml prefilled syringe.

### Methods

Samples were stored at 37° C. for up to 21 days. After 14 days and 21 days, samples were analysed to determine the HMWP and cagrilintide purity levels.

Levels of covalently bound HMWP were quantified using size exclusion chromatography (SEC). Samples were analysed using a WATERS HMWP column (7.8×300 mm) with an isocratic elution consisting of 500 mM sodium chloride, 10 mM sodium dihydrogen phosphate monohydrate, 5 mM ortho-phosphate and 50% (v/v) isopropanol. Chromatography was conducted with UV detection (215 nm) at 50° C. using a 10 μl injection volume and a flow rate of 0.5 ml/min. HMWP was quantified as being the area of all components eluting before the main peak divided by the area of the main peak×100%.

Cagrilintide purity was determined using reversed phase ultra-high performance liquid chromatography (RP-UHPLC). Samples were analysed using a Kinetex C18, 1.7 μm, 100 Å, column (2.1×150 mm) with a gradient elution of eluent A consisting of 90% v/v 0.09 M phosphate solution, pH 3.6 and 10% v/v acetonitrile, and eluent B consisting of 60% v/V acetonitrile and 20% v/v isopropanol. Chromatography was conducted with UV detection (215 nm) at 60° C. using 2-7.5 μl injection volume and a flow rate of 0.25 ml/min. Purity was evaluated as the area of the main peak divided by the area of all peaks×100%.

TABLE 2
Chemical purity (%) of cagrilintide
at pH 4.0 and 6.0 with and without

| | | | Levels of | | | Purity (%) | | |
|---|---|---|---|---|---|---|---|---|
| Cagril- | | HP-B- | HMWP (%) as | | | of cagrilintide as | | |
| intide | | CD | a function of time | | | a function of time | | |
| formu- | | (MS: | (days) at 37° C. | | | (days) at 37° C. | | |
| lation | pH | 0.92)¹ | 0 | 14 | 21 | 0 | 14 | 21 |
| 1 | 4.0 | 0% w/v | 0.0% | 0.2% | 0.2% | 90.4% | 88.2% | 87.4% |
| 2 | 6.0 | 0% w/v | 0.1% | 7.7% | 9.8% | 90.1% | 74.0% | 67.1% |
| 3 | 6.0 | 25% w/v | 0.1% | 0.3% | 0.5% | 90.2% | 79.9% | 75.3% |

¹MS: molar substitution, corresponds to hydroxypropyls per glucose unit

### Concluding Remarks

Table 2 shows that when cagrilintide was stored at 37° C. and at a pH of 4.0, very little HMWP was formed and only a minor decrease in cagrilintide purity was seen. In contrast, when the pH was 6.0 the rate of HMWP formation and decrease in cagrilintide purity accelerated. Surprisingly, this rapid chemical degradation was counteracted by the addition of HP-B-CD to the formulation, making it possible to formulate cagrilintide at pH 6.

### Example 2: Effect of Hp-B-Cd on Semaglutide Physical Stability

This example demonstrates the ability of HP-B-CD to physically stabilise semaglutide, which has a propensity to form peptide fibrils. The effect was evident when semaglutide was formulated at suboptimal pH.

### Composition

The compositions of semaglutide formulations 1, 2 and 3 are shown in table 3.

TABLE 3
Composition of semaglutide formulations 1, 2 and 3

| | Semaglutide formulation | | |
|---|---|---|---|
| Ingredient | 1 | 2 | 3 |
| Semaglutide drug | 4.8 | 4.8 | 4.8 |
| substance (mg/ml) | | | |
| HP-B-CD | 0 | 25 | 0 |
| KLEPTOSE ® | | | |
| (Roquette) (% w/v) | | | |
| (Average MS: 0.92, | | | |
| MS range: 0.81-0.99)¹ | | | |
| Citrate, 1 H₂O ² (mM) | 3.7 | 3.7 | 0.9 |
| Disodium hydrogen | 12.6 | 12.6 | 18.2 |
| phosphate, 2 H₂O ² | | | |
| (mM) | | | |
| HCl | q.s. | q.s. | q.s. |
| NaOH | q.s. | q.s. | q.s. |
| WFI | To make 1 ml | To make 1 ml | To make 1 ml |
| pH | 6.0 | 6.0 | 7.4 |

¹MS: molar substitution, corresponds to hydroxypropyls per glucose unit
² Different buffer concentrations across formulations are used to ensure buffering at different pH

¹ MS: molar substitution, corresponds to hydroxypropyls per glucose unit
² Different buffer concentrations across formulations are used to ensure buffering at different pH

### Preparation Process

Formulations were prepared as described in example 1.

### Methods

The propensity of semaglutide to aggregate and form peptide fibrils, parameters used to quantify physical stability, was measured using a Thioflavin T (ThT) fluorescence stress assay. The analysis for presence of peptide fibrils is based on the fluorescence characteristics of the ThT probe, which displays low fluorescence in the unbound state/native peptide-bound state but high fluorescence when bound to peptide fibrils as well as a red shift in the wavelength of maximum fluorescence upon fibril binding.

Two samples were pooled and 1400 μl sample was added to 28 μl 1 mM ThT stock solution, of which 200 μl was then transferred to 6 different wells on a 96 well microtiter plate with a glass bead in. The assay was run with double orbital shaking and a speed of 300 rpm at 40° C. for 169 hours on a BMG CLARIOstar fluorescence plate reader equipped with monochromators for both excitation and emission using 450 nm and 480 nm, respectively. The lag time was measured from the start of the experiment until fibrillation occurs, shown as an increase in ThT fluorescence.

TABLE 4
Physical stability for semaglutide at pH 6.0 and 7.4

| Semaglutide | | HP-B-CD | | Lag time | |
|---|---|---|---|---|---|
| formulation | pH | (MS: 0.92)³ | | until fibrillation | |
| 1 | 6.0 | 0% | w/v | 2.35 | hours ¹ |
| 2 | 6.0 | 25% | w/v | >169 | hours ^{1, 2} |
| 3 | 7.4 | 0% | w/v | >169 | hours ^{1, 2} |

¹ Result is the mean of 6 replicates
² Fibrillation was not observed in any of the 6 replicates within the 169 hours' duration of the experiment
³MS: molar substitution, corresponds to hydroxypropyls per glucose unit

### Concluding Remarks

The semaglutide formulations were subjected to shear stress-inducing conditions and the propensity of semaglutide to form peptide fibrils was measured. Surprisingly, the presence of HP-B-CD was found to inhibit semaglutide peptide fibril formation. When semaglutide was formulated at pH 6 and in the absence of HP-B-CD (semaglutide formulation 1), fibrillation occurred after 2.35 hours; that is, semaglutide was not physically stable. However, when semaglutide was formulated at pH 6 and in the presence of HP-B-CD (semaglutide formulation 2), no fibrillation was observed throughout the duration of the experiment; that is, semaglutide was physically stable. Furthermore, the physical stability of semaglutide, when formulated at pH 6 and in the presence of HP-B-CD (semaglutide formulation 2), was found comparable to the physical stability of semaglutide when formulated in the absence of HP-B-CD but at its optimal formulation conditions in terms of pH 7.4 (semaglutide formulation 3).

### Example 3: Effect of Hp-B-Cd on Semaglutide Chemical Stability

This example demonstrated the ability of HP-B-CD to chemically stabilise semaglutide, chemical stability being measured in terms of semaglutide purity and semaglutide-related high molecular weight protein (HMWP).

### Composition

The same formulations were used as in example 2.

### Preparation Process

Formulations were prepared as described in example 1.

### Methods

Levels of HMWP and semaglutide purity were determined after 0 days, 14 days, and 21 days' storage at 37° C.

Semaglutide purity was determined using reversed phase high performance liquid chromatography (RP-HPLC) where the samples were analysed using a Kinetex C18, 2.6 μm column (4.6×150 mm) with a gradient elution of eluent A consisting of 90% v/v 0.09 M phosphate solution, pH 3.6 and 10% v/v acetonitrile, and eluent B consisting of 60% v/v acetonitrile and 20% v/v isopropanol. Chromatography was conducted with UV detection (210 nm) at 30° C. using a 10-100 μl injection volume and a flow rate of 0.7 ml/min. Purity was quantified as being the area of the main peak divided by the area of all peaks×100%.

The level of covalently bound HMWP was determined using size exclusion chromatography (SEC). Samples were analysed using a Waters SEC 1.7 μm column (4.6×150 mm) with an isocratic elution consisting of 300 mM sodium chloride, 10 mM sodium dihydrogen phosphate, 5 mM ortho-phosphate and 50% v/v 2-propanol. Chromatography was conducted with UV detection (280 nm) at 50° C. using a 1-10 μl injection volume and a flow rate of 0.3 ml/min. HMWP was quantified as being the area of all components eluting before the main peak divided by the area of the main peak×100%.

TABLE 5
Semaglutide purity at suboptimal and optimal pH

| | | | Purity (%) of | | | Levels of | | |
|---|---|---|---|---|---|---|---|---|
| | | HP-B- | semaglutide as | | | HMWP (%) as a | | |
| | | CD | a function of time | | | function of time | | |
| Semaglutide | | (MS: | (days) at 37° C. | | | (days) at 37° C. | | |
| formulation | pH | 0.92)¹ | 0 | 14 | 21 | 0 | 14 | 21 |
| 1 | 6.0 | 0% w/v | 96.89% | 94.58% | 93.48% | 0.12% | 0.25% | 0.35% |
| 2 | 6.0 | 25% w/v | 96.51% | 94.53% | 93.73% | 0.04% | 0.11% | 0.11% |
| 3 | 7.4 | 0% w/v | 96.82% | 95.56% | 94.81% | 0.12% | 0.32% | 0.40% |

¹MS: molar substitution, corresponds to hydroxypropyls per glucose unit

### Concluding Remarks

The results in table 5 show that the chemical purity of semaglutide decreased over time. The chemical purity of semaglutide decreased more rapidly when it was formulated at pH 6.0 (semaglutide formulation 1) than when it was formulated at its optimal pH 7.4 (semaglutide formulation 3). Surprisingly, HP-B-CD improved the chemical stability of semaglutide (in terms of purity decline and HMWP formation) when it was formulated at pH 6.0 (semaglutide formulation 2).

### Example 4: Effect of the Molar Substution of Hp-B-Cd on Cagrilintide and Semaglutide Co-Formulation Physical Stability

This example shows the effect of HP-B-CD molar substitution on the physical stability of cagrilintide and semaglutide.

### Composition

The compositions of co-formulation 1 and co-formulation 2 are shown in table 6.

TABLE 6
Composition of co-formulation 1 and co-formulation 2

Co-formulation

| Ingredient | 1 | 2 |
|---|---|---|
| Cagrilintide drug | 3.2 | 3.2 |
| substance (mg/ml) | | |
| Semaglutide drug | 3.2 | 3.2 |
| substance (mg/ml) | | |
| HP-B-CD | 25 | — |
| KLEPTOSE ® | | |
| (Roquette) (% w/v) | | |
| (Average MS: 0.92, | | |
| MS range: 0.81-0.99)¹ | | |
| HP-B-CD | — | 25 |
| KLEPTOSE ® | | |
| (Roquette) (% w/v) | | |
| (Average MS: 0.62, | | |
| MS range: 0.58-0.68)¹ | | |
| Citrate, 1 H₂O (mM) | 3 | 3 |
| HCl | q.s. | q.s. |
| NaOH | q.s. | q.s. |
| WFI | To make 1 ml | To make 1 ml |
| pH | 5.7 | 5.7 |

¹MS: molar substitution, corresponds to hydroxypropyls per glucose unit

### Preparation Process

Formulations were prepared as described in example 1.

### Methods

All samples were stored at stressed conditions, defined as:
Duration: 28 days
Temperature: 30° C.±2° C.
Stress condition: During storage, samples were inverted 360° to simulate patient use out of refrigerated storage. The rotations were performed 20 times three days every week, and 40 times two days every week.

The number of sub-visible particles present quantifies the physical stability of cagrilintide and semaglutide combined and were obtained by means of micro-flow imaging (MFI, see e.g. Sharma, D. K. et al. AAPS J. (2010), 12:455-464 for principles of the MFI technique). The following procedure was employed for each analysed syringe sample: The experiment was performed at ambient temperature. The liquid from each syringe was taken out by first removing the plunger and then pipetting the liquid into the sample container. The sample was transferred to a 96 deep-well plate which was inserted into the sample handling unit (Bot1) of a Protein Simple MFI™ 5200 apparatus equipped with a standard Protein Simple MFI™ 100 μm flow cell. The sample was analysed by standard MFI system settings implying that the liquid was pipetted into a reservoir connected to a flow cell, the liquid was illuminated by a 10 LED light source (470 nm), and a digital camera (via magnification optics) recorded the contents of the flow cell as bright field images throughout the experiment. Data acquisition was accomplished using Protein Simple MVSS software. The recorded image stream from the entire run was processed by validated Novo Nordisk proprietary software MFI Data Validator whereby the number (normalised to counts per ml analysed liquid) of individual particles was obtained and presented by size; >5 μm, >10 μm, and >25 μm which are standard size ranges for sub-visible particles. Note that the number of particles >5 μm includes all particles greater than 5 μm in diameter (>5 μm, >10 μm and >25 μm) and the number of particles >10 μm includes all particles greater than 10 μm in diameter (>10 μm and >25 μm). The particle size is defined as the equivalent circular diameter (ECD).

The presence of amyloid peptide fibrils was analysed with a Thioflavin-T (ThT) fluorescence assay. The experiment was performed at 25° C. The liquid from each syringe was taken out by first removing the plunger and then pipetting the liquid into the sample container. Subsequently 500 μl of the sample was mixed with approximately 9 μl of ThT stock solution in a separate sample container, to give a final ThT concentration of 20 μM. The sample was left to incubate in the dark for 25 min at ambient temperature. 200 μl sample was transferred to a well in a 96-well microtiter plate. Samples were measured on a BMG CLARIOstar fluorescence plate reader equipped with monochromators for both excitation and emission using 440 nm and 470-550 nm, respectively.

Data acquisition was accomplished using CLARIOstar Control software. Emission maximum in the present assay was observed to occur at a wavelength of approximately 485 nm; the result for each analysis was therefore reported as the ThT fluorescence at 485 nm, expressed in Relative Fluorescence Units (RFU).

TABLE 8
Co-formulation physical stability with HP-B-CD with a high or
medium molar substitution

| Co- | | Number of sub-visible particles | | | |
|---|---|---|---|---|---|
| formu- | HP-B-CD average molar | as a function of time (days) | | | |
| lation | substitution | 0 | 14 | 21 | 28 |

Particle size >5 μm

| | | | | | |
|---|---|---|---|---|---|
| 1 | HP-B-CD (MS: 0.92)¹ | 459 | 334 | 1754 | 22433 |
| 2 | HP-B-CD (MS: 0.62)¹ | 256 | 379 | 715 | 245 |

Particle size >10 μm

| | | | | | |
|---|---|---|---|---|---|
| 1 | HP-B-CD (MS: 0.92)¹ | 52 | 27 | 602 | 10598 |
| 2 | HP-B-CD (MS: 0.62)¹ | 39 | 39 | 132 | 122 |

Particle size >25 μm

| | | | | | |
|---|---|---|---|---|---|
| 1 | HP-B-CD (MS: 0.92)¹ | 0 | 0 | 2 | 644 |
| 2 | HP-B-CD (MS: 0.62)¹ | 6 | 0 | 8 | 4 |

ThT fluorescence at 485 nm

| | | | | | |
|---|---|---|---|---|---|
| 1 | HP-B-CD (MS: 0.92)¹ | 3357 | 3538 | 3588 | 13640 |
| 2 | HP-B-CD (MS: 0.62)¹ | 3784 | 3720 | 3967 | 3463 |

Results for number of sub-visible particles are the mean of 3 replicates and has been rounded to nearest integer value
¹MS: molar substitution, corresponds to hydroxypropyls per glucose unit

### Concluding Remarks

The results in table 7 show that fewest particles were generated in co-formulation 2 containing HP-B-CD (Average MS: 0.62). Furthermore, during the 28 days' duration of the experiment, no increase in the number of sub-visible particles or ThT fluorescence was seen in the case of co-formulation 2.

In contrast, an increase in sub-visible particle count was seen after 21 days, and an increase in ThT fluorescence was seen after 28 days, in the case of co-formulation 1 containing HP-B-CD (Average MS: 0.92).

In this otherwise identical, citrate-buffered co-formulation of semaglutide and cagrilintide, the co-formulation (2) comprising HP-B-CD (Average MS: 0.62) was more physically stable than the co-formulation (1) comprising HP-B-CD (Average MS: 0.92).

### Example 5: Effect of Hydroxypropyl-B-Cyclodextrin Concentration on Semaglutide Chemical Stability

This example shows the concentration-dependent effect of HP-B-CD on the chemical stability of semaglutide.

### Composition

The composition of co-formulation 3, co-formulation 4, and co-formulation 5 are shown in table 8.

TABLE 8
Composition of co-formulation 3, co-formulation 4, and co-formulation 5

| | Co-formulation | | |
|---|---|---|---|
| Ingredient | 3 | 4 | 5 |
| Cagrilintide drug substance | 3.2 | 3.2 | 3.2 |
| (mg/ml) | | | |
| Semaglutide drug substance | 3.2 | 3.2 | 3.2 |
| (mg/ml) | | | |
| HP-B-CD | 11.25 | 12.5 | 15 |
| KLEPTOSE® (Roquette) | | | |
| (% w/V) | | | |
| (Average MS: 0.62, MS range: | | | |
| 0.58-0.68)¹ | | | |
| L-Histidine (mM) | 6 | 6 | 6 |
| Sorbitol² (mg/ml) | 26 | 24 | 20 |
| Polysorbate 80 (mg/ml) | 0.05 | 0.05 | 0.05 |
| HCl | q.s. | q.s. | q.s. |
| NaOH | q.s. | q.s. | q.s. |
| WFl | To make | To make | To make |
| | 1 ml | 1 ml | 1 ml |
| pH | 5.7 | 5.7 | 5.7 |

¹ MS: molar substitution, corresponds to hydroxypropyls per glucose unit
² Different sorbitol concentrations are needed to obtain isotonicity because of the varying HP-B-CD concentrations tested

### Preparation

Formulations were prepared as described in example 1.

### Method

Samples were stored at 37° C. for 28 days at which samples were analysed to determine the chemical purity of semaglutide after 14, 21, and 28 days.

The purity of semaglutide was determined using reversed phase ultra-high performance liquid chromatography (RP-UHPLC), where the samples were analysed using a Waters Acquity phenyl-hexyl 1.7 μm column (2.1×150 mm) with a gradient elution of eluent A consisting of 0.09% TFA in MQ water, and eluent B consisting of 0.09% TFA in MQ water 0.09% TFA in 80% acetonitrile in MQ water. Chromatography was conducted with UV detection (215 nm) at 62° C. using 2-14 μl injection volume and a flow rate of 0.25 ml/min. Purity was evaluated as the area of the main peak of semaglutide divided by the area of all related peaks ×100%.

Note that, in other experiments, the same method was used to determine cagrilintide purity.

TABLE 9
Chemical purity (%) for semaglutide with different HP-B-CD
concentrations

| | | Semaglutide purity (%) as a function of | | | |
|---|---|---|---|---|---|
| Co- | HP-B-CD | time (days) at 37° C. | | | |
| formulation | (MS: 0.62)¹ | 0 | 14 | 21 | 28 |
| 3 | 11.25% w/V | 96.90% | 90.72% | 88.65% | 87.52% |
| 4 | 12.5% w/V | 96.87% | 92.90% | 91.25% | 89.81% |
| 5 | 15% w/V | 96.83% | 93.61% | 92.92% | 91.67% |

¹MS: molar substitution, corresponds to hydroxypropyls per glucose unit

### Concluding Remarks

The results in table 9 show that the chemical stability and thus purity of semaglutide also depended on HP-B-CD concentration. Semaglutide remained chemically stable in all of the co-formulations (comprising 11.25-15% w/v HP-B-CD). However, semaglutide chemical stability and thus purity was highest when the co-formulation comprised 15% w/v HP-B-CD.

### Example 6: Effect of Different Tonicity Agents on Co-Formulation Physical Stability

This example shows the stabilising effect of different tonicity agents on the physical stability of otherwise identical cagrilintide and semaglutide co-formulations.

Composition The compositions of co-formulation 6 to co-formulation 12 are shown in in table 10.

TABLE 10
Composition of co-formulation 6 to co-formulation 12

| | Co-formulation | | | | | | |
|---|---|---|---|---|---|---|---|
| Ingredient | 6 | 7 | 8 | 9 | 10 | 11 | 12 |
| Cagrilintide drug | 3.2 | 3.2 | 3.2 | 3.2 | 3.2 | 3.2 | 3.2 |
| substance (mg/ml) | | | | | | | |
| Semaglutide drug | 3.2 | 3.2 | 3.2 | 3.2 | 3.2 | 3.2 | 3.2 |
| substance (mg/ml) | | | | | | | |
| HP-B-CD | 10 | 10 | 10 | 10 | 10 | 10 | 10 |
| KLEPTOSE ® | | | | | | | |
| (Roquette) (% w/v) | | | | | | | |
| (Average MS: 0.62, | | | | | | | |
| MS range: 0.58-0.68)¹ | | | | | | | |
| L-Histidine (mM) | 6 | 6 | 6 | 6 | 6 | 6 | 6 |
| Polysorbate 20 | 0.1 | 0.1 | 0.1 | 0.1 | 0.1 | 0.1 | 0.1 |
| (mg/ml) | | | | | | | |
| Glycerol (mg/ml) | — | 16 | — | — | — | — | — |
| Sorbitol (mg/ml) | — | — | 34 | — | — | — | — |
| Mannitol (mg/ml) | — | — | — | 34 | — | — | — |
| Trehalose (mg/ml) | — | — | — | — | 63 | — | — |
| Sucrose (mg/ml) | — | — | — | — | — | 63 | — |
| NaCl (mg/ml) | — | — | — | — | — | — | 6.0 |
| HCl | q.s. | q.s. | q.s. | q.s. | q.s. | q.s. | q.s. |
| NaOH | q.s. | q.s. | q.s. | q.s. | q.s. | q.s. | q.s. |
| WFI | To | To | To | To | To | To | To |
| | make | make | make | make | make | make | make |
| | 1 ml | 1 ml | 1 ml | 1 ml | 1 ml | 1 ml | 1 ml |
| pH | 5.7 | 5.7 | 5.7 | 5.7 | 5.7 | 5.7 | 5.7 |

¹MS: molar substitution, corresponds to hydroxypropyls per glucose unit

### Preparation Process

Formulations were prepared as described in example 1.

### Methods

All samples were stored for at stressed condition defined as:
Duration: 18 days
Temperature: 37° C.±2° C. . . .
Stress condition: During storage, samples were invented 360° to simulate patient use out of refrigerated storage. The rotations were performed 100 times, five days a week.

The number of sub-visible particles was quantified as described in example 4.

TABLE 11
The effect of different tonicity agents
on co-formulation physical stability

| | | Number of sub-visible particles | | | | |
|---|---|---|---|---|---|---|
| Co- | Tonicity | as a function of time (days) | | | | |
| formulation | agent | 0 | 7 | 11 | 14 | 18 |

Particle size >5 μm

| | | | | | | |
|---|---|---|---|---|---|---|
| 6 | None | 256 | 385 | 429 | 952 | 1296 |
| 7 | Glycerol | 292 | 241 | 292 | 466 | 691 |
| 8 | Sorbitol | 212 | 341 | 340 | 517 | 345 |
| 9 | Mannitol | 94 | 438 | 177 | 245 | 360 |
| 10 | Trehalose | 121 | 155 | 213 | 504 | 472 |
| 11 | Sucrose | 59 | 104 | 140 | 539 | 749 |
| 12 | NaCl ¹ | 36 | 946 | — | — | — |

Particle size >10 μm

| | | | | | | |
|---|---|---|---|---|---|---|
| 6 | None | 4 | 27 | 96 | 216 | 377 |
| 7 | Glycerol | 10 | 23 | 23 | 48 | 106 |
| 8 | Sorbitol | 6 | 54 | 42 | 36 | 73 |
| 9 | Mannitol | 2 | 23 | 12 | 8 | 50 |
| 10 | Trehalose | 4 | 10 | 40 | 50 | 69 |
| 11 | Sucrose | 2 | 8 | 13 | 142 | 253 |
| 12 | NaCl ¹ | 2 | 403 | — | — | — |

Particle size >25 μm

| | | | | | | |
|---|---|---|---|---|---|---|
| 6 | None | 0 | 4 | 4 | 27 | 75 |
| 7 | Glycerol | 0 | 0 | 4 | 6 | 4 |
| 8 | Sorbitol | 0 | 0 | 2 | 4 | 0 |
| 9 | Mannitol | 0 | 0 | 0 | 0 | 0 |
| 10 | Trehalose | 0 | 0 | 0 | 0 | 4 |
| 11 | Sucrose | 0 | 0 | 2 | 15 | 55 |
| 12 | NaCl ¹ | 0 | 106 | — | — | — |

Results are the mean of 2 replicates and has been rounded to nearest integer value
(—) Sampling not performed
¹ For co-formulation 12 with NaCl, sampling was discontinued earlier than for the other formulations because of the rapid increase in sub-visible particle counts.

### Concluding Remarks

The results in table 11 show that the sub-visible particle count increased most rapidly in the co-formulation comprising NaCl as tonicity agent (co-formulation 12). After 7 days, the particle count vastly exceeded the particle count determined for the other co-formulations. Therefore, sampling for the analysis of number of sub-visible particles was discontinued for the NaCl-containing co-formulation after 7 days.

After 14 days an increase in sub-visible particle count was seen in the co-formulations containing glycerol and sucrose and the two co-formulations were deemed comparable regarding physical stability. The particle count remained lowest in the co-formulations that contained mannitol, sorbitol or trehalose. In these co-formulations, virtually no increase in the number of sub-visible particles was seen during the 18 days that the co-formulations were stored at stressed conditions.

Of the co-formulations tested, those comprising mannitol, sorbitol or trehalose as tonicity agent remained the most stable over time.

### Example 7: Effect of Different Surfactants on Co-Formulation Physical Stability

This example shows the effect of different surfactants on the physical stability of otherwise identical cagrilintide and semaglutide co-formulations.

### Composition

The compositions of co-formulation 13, co-formulation 14, and co-formulation 15 are shown in table 12.

TABLE 12
Composition of co-formulation 13, co-formulation 14, and co-formulation

| | Co-formulation | | |
|---|---|---|---|
| Ingredient | 13 | 14 | 15 |
| Cagrilintide drug substance | 3.2 | 3.2 | 3.2 |
| (mg/ml) | | | |
| Semaglutide drug substance | 3.2 | 3.2 | 3.2 |
| (mg/ml) | | | |
| HP-B-CD | 10 | 10 | 10 |
| KLEPTOSE® (Roquette) | | | |
| (% w/V) | | | |
| (Average MS: 0.62, MS range: | | | |
| 0.58-0.68)¹ | | | |
| L-Histidine (mM) | 6 | 6 | 6 |
| Polysorbate 20 (mg/ml) | 0.05 | — | — |
| Polysorbate 80 (mg/ml) | — | 0.05 | — |
| Poloxamer 188 (mg/ml) | — | — | 0.5 |
| HCl | q.s. | q.s. | q.s. |
| NaOH | q.s. | q.s. | q.s. |
| WFl | To make | To make | To make |
| | 1 ml | 1 ml | 1 ml |
| pH | 5.7 | 5.7 | 5.7 |

¹MS: molar substitution, corresponds to hydroxypropyls per glucose unit

### Preparation Process

Formulations were prepared as described in example 1.

### Methods

All samples were stored for at stressed condition defined as:
Duration: 17 days
Temperature: 37° C.±2° C. . . .
Stress condition: During storage, samples were invented 360° to simulate patient use out of refrigerated storage. The rotations were performed 100 times, five days every week.

The number of sub-visible particles was quantified as described in example 4.

TABLE 13
The effect of different surfactants on
co-formulation physical stability

| | | Number of sub-visible particles as | | | | |
|---|---|---|---|---|---|---|
| Co- | | a function of time point (days) | | | | |
| formulation | Surfactant | 0 | 7 | 10 | 14 | 17 |

Particle size >5 μm

| | | | | | | |
|---|---|---|---|---|---|---|
| 13 | Polysorbate | 464 | 304 | 396 | 772 | 2803 |
| | 20 | | | | | |
| 14 | Polysorbate | 566 | 292 | 520 | 286¹ | 348 |
| | 80 | | | | | |
| 15 | Poloxamer | 1235 | 1278 | 2783 | 940¹ | 1704 |
| | 188 | | | | | |

Particle size >10 μm

| | | | | | | |
|---|---|---|---|---|---|---|
| 13 | Polysorbate | 23 | 23 | 43 | 143 | 297 |
| | 20 | | | | | |
| 14 | Polysorbate | 19 | 31 | 76 | 57¹ | 48 |
| | 80 | | | | | |
| 15 | Poloxamer | 100 | 245 | 301 | 290¹ | 523 |
| | 188 | | | | | |

Particle size >25 μm

| | | | | | | |
|---|---|---|---|---|---|---|
| 13 | Polysorbate | 0 | 2 | 6 | 11 | 13 |
| | 20 | | | | | |
| 14 | Polysorbate | 0 | 0 | 0 | 4¹ | 0 |
| | 80 | | | | | |
| 15 | Poloxamer | 0 | 13 | 17 | 61¹ | 52 |
| | 188 | | | | | |

Results are the mean of 2 replicates and has been rounded to nearest integer value
¹Only one replicate was performed

### Concluding Remarks

Co-formulation 14 contained the lowest number of sub-visible particles when stored for 17 days under stressed conditions. In co-formulation 13, containing polysorbate 20, an increase in sub-visible particles was observed after 14 days, while in co-formulation 15 containing poloxamer 188 sub-visible particles are formed after 7 days at stressed conditions. It is evident that the co-formulation containing polysorbate 80 was the most stable and that the co-formulation containing polysorbate 20 was also acceptably stable.

### Example 8: Effect of Different Buffer Substances on Co-Formulation Physical Stability

This example shows that the buffer substance has an effect on the physical stability of an otherwise identical cagrilintide and semaglutide co-formulation.

### Composition

The composition of co-formulation 1 and co-formulation 16 are shown in table 14.

TABLE 14
Composition of co-formulation 1 and co-formulation 16

| | Co-formulation | |
|---|---|---|
| Ingredient | 1 | 16 |
| Cagrilintide drug substance (mg/ml) | 3.2 | 3.2 |
| Semaglutide drug substance (mg/ml) | 3.2 | 3.2 |
| HP-B-CD | 25 | 25 |
| KLEPTOSE® (Roquette) (% w/V) | | |
| (Average MS: 0.92, MS range: 0.81-0.99)¹ | | |
| Citrate, 1 H2O (mM) | 3 | — |
| L-Histidine (mM) | — | 6 |
| HCl | q.s. | q.s. |
| NaOH | q.s. | q.s. |
| WFl | To make | To make |
| | 1 ml | 1 ml |
| pH | 5.7 | 5.7 |

¹MS: molar substitution, corresponds to hydroxypropyls per glucose unit

### Preparation Process

Formulations were prepared as described in example 1.

### Methods

All samples were stored for at stressed condition defined as:
Duration: 21 days
Temperature: 37° C.±2° C.
Stress condition: During storage, samples were invented 360° to simulate patient use out of refrigerated storage. The rotations were performed 100 times, five days every week.
   The number of sub-visible particles was quantified as described in example 4.

TABLE 15
The effect of buffer substance
on co-formulation physical stability

| | | Number of sub-visible particles | | | | | |
|---|---|---|---|---|---|---|---|
| Co- | Buffer | as a function of time (days) | | | | | |
| formulation | substance | 0 | 7 | 11 | 14 | 18 | 21 |

Particle size >5 μm

| | | | | | | | |
|---|---|---|---|---|---|---|---|
| 1 | Citrate | 459 | 668 | 408 | 409 | 4717 | 3115 |
| 16 | Histidine | 386 | 752 | 569 | 571 | 2495 | 2392 |

Particle size >10 μm

| | | | | | | | |
|---|---|---|---|---|---|---|---|
| 1 | Citrate | 52 | 117 | 55 | 125 | 2114 | 1446 |
| 16 | Histidine | 68 | 80 | 103 | 151 | 969 | 760 |

Particle size >25 μm

| | | | | | | | |
|---|---|---|---|---|---|---|---|
| 1 | Citrate | 0 | 0 | 0 | 16 | 542 | 332 |
| 16 | Histidine | 2 | 0 | 4 | 13 | 162 | 117 |

Results are the mean of 2 replicates and has been rounded to nearest integer value

### Concluding Remarks

Until day 14 of having been stored at stressed conditions, the physical stability of the two co-formulations was similar and acceptable. However, after 18 days, the number of sub-visible particles in the citrate-buffered co-formulation (co-formulation 1) was much greater than that in the histidine-buffered co-formulation (co-formulation 16). The histidine-buffered co-formulation 16 was the most stable.

### Example 9: Effect of Different Buffers Concentration on Co-Formulation Chemical Stability

This example shows the effect of buffer concentration on the chemical stability of otherwise identical co-formulations.

### Composition

The compositions of co-formulation 17 and co-formulation 18 are shown in table 16.

TABLE 16
Composition of co-formulation 17 and co-formulation 18

| | Co-formulation | |
|---|---|---|
| Ingredient | 17 | 18 |
| Cagrilintide drug substance (mg/ml) | 3.2 | 3.2 |
| Semaglutide drug substance (mg/ml) | 3.2 | 3.2 |
| HP-B-CD | 10 | 10 |
| KLEPTOSE® (Roquette) (% w/v) | | |
| (Average MS: 0.62, MS range: | | |
| 0.58-0.68)¹ | | |
| L-Histidine (mM) | 6 | 20 |
| HCl | q.s. | q.s. |
| NaOH | q.s. | q.s. |
| WFl | To make 1 ml | To make 1 ml |
| pH | 5.7 | 5.7 |

¹MS: molar substitution, corresponds to hydroxypropyls per glucose unit

### Preparation Process

Formulations were prepared as described in example 1.

### Methods

Samples were stored at 30° C. for 21 days and analysed to determine the chemical purity of cagrilintide after 7, 14, and 21 days. Purity of cagrilintide was determined as described in example 5 (for semaglutide).

TABLE 17
The effect of buffer concentration on cagrilintide chemical stability
in co-formulation

| | | Cagrilintide purity (%) as a function | | | |
|---|---|---|---|---|---|
| Co- | Buffer | of time (days) at 30° C. | | | |
| formulation | concentration | 0 | 7 | 14 | 21 |
| 17 | Histidine: 6 mM | 89.2% | 88.2% | 86.5% | 84.2% |
| 18 | Histidine: 20 mM | 89.2% | 87.9% | 86.0% | 83.5% |

### Concluding Remarks

The results in table 17 show that both co-formulations were stable. However, the chemical purity of cagrilintide was highest in co-formulation 17. The purity of cagrilintide decreased more rapidly over time when the histidine concentration was 20 mM.

### Example 10: Effect of Different Buffers Concentration on Co-Formulation Physical Stability

This example shows the effect of histidine buffer concentration on co-formulation physical stability.

### Composition

The compositions of the tested co-formulations are as shown in table 16

### Preparation Process

Formulations were prepared as described in example 1.

Methods All samples were stored for at stressed condition defined as:
Duration: 18 days
Temperature: 37° C.±2° C.
Stress condition: During storage, samples were invented 360° to simulate patient use out of refrigerated storage. The rotations were performed 100 times, five days every week.
   The number of sub-visible particles was quantified as described in example 4.

TABLE 18
The effect of buffer concentration
on co-formulation physical stability

| | | Number of sub-visible particles | | | | |
|---|---|---|---|---|---|---|
| Co- | Buffer | as a function of time point (days) | | | | |
| formulation | concentration | 0 | 7 | 10 | 14 | 18 |

Particle size >5 μm

| | | | | | | |
|---|---|---|---|---|---|---|
| 17 | Histidine: 6 mM | 239 | 392 | 165 | 189 | 266 |
| 18 | Histidine: 20 mM | 50 | 262 | 197 | 1029 | 926 |

Particle size >10 μm

| | | | | | | |
|---|---|---|---|---|---|---|
| 17 | Histidine: 6mM | 4 | 8 | 15 | 17 | 46 |
| 18 | Histidine: 20 mM | 2 | 17 | 17 | 461 | 423 |

Particle size >25 μm

| | | | | | | |
|---|---|---|---|---|---|---|
| 17 | Histidine: 6mM | 0 | 0 | 2 | 0 | 2 |
| 18 | Histidine: 20 mM | 0 | 0 | 0 | 120 | 104 |

Results are the mean of 2 replicates and has been rounded to nearest integer value.

### Concluding Remarks

The difference in the physical stability of co-formulations 17 and 18 became most apparent after 14 days. The data in table 18 show that the number of sub-visible particles seen in co-formulation 18 (containing 20 mM histidine) was greater than the number of sub-visible particles seen in co-formulation 17 (containing 6 mM histidine). That is, the co-formulation comprising 6 mM histidine was the most physically stable.

### Example 11: Effect of Hp-B-Cd Concentration on Subcutaneous Tolerance Upon Subcutaneous Injection

This example shows the concentration dependent effect of HP-B-CD on the subcutaneous tissue upon subcutaneous injection.

### Composition

The compositions of the tested co-formulation vehicles prepared with varying HP—B-CD concentrations are shown in table 19.

TABLE 19
Composition of co-formulation vehicles containing varying
concentrations of HP-B-CD

| | Co-formulation vehicle | | | | |
|---|---|---|---|---|---|
| Ingredient | 1 | 2 | 3 | 4 | 5 |
| HP-B-CD | 10 | 12.5 | 15 | 17.5 | 20 |
| KLEPTOSE® (Roquette) | | | | | |
| (% w/V) | | | | | |
| (Average MS: 0.62, MS range: | | | | | |
| 0.58-0.68)¹ | | | | | |
| L-Histidine (mM) | 6 | 6 | 6 | 6 | 6 |
| Sorbitol² (mg/ml) | 28 | 24 | 20 | 16 | 12 |
| Polysorbate 80 (mg/ml) | 0.05 | 0.05 | 0.05 | 0.05 | 0.05 |
| HCl | q.s. | q.s. | q.s. | q.s. | q.s. |
| NaOH | q.s. | q.s. | q.s. | q.s. | q.s. |
| WFl | To 1 | To 1 | To 1 | To 1 | To 1 |
| | ml | ml | ml | ml | ml |
| pH | 6.0 | 6.0 | 6.0 | 6.0 | 6.0 |

¹ MS: molar substitution, corresponds to hydroxypropyls per glucose unit.
² Sorbitol concentration varies with varying HP-B-CD concentration to maintain isotonic conditions.

### Preparation Process

Formulations were prepared as described in example 1 except that addition of active pharmaceutical ingredients was abstained from.

### Method

The local (subcutaneous) tolerance upon subcutaneous administration of formulations containing HP-B-CD was studied in 5 live Landrace×Yorkshirex Duroc (LYD) pigs by evaluation of the resulting skin lesions 6 days (necropsy) after subcutaneous administration of 600 μl using syringes equipped with 25 G sized needles and 5 mm stoppers. Skin samples sized 2×2 cm were collected at necropsy, fixed in neutral buffered formalin, trimmed using multi-knife, embedded in paraffin, cut in 4 μm thin sections, mounted on glass slides and subsequently hematoxylin-eosin (HE) stained. The level of subcutaneous tissue necrosis was assessed using a light microscope and scored on a numerical scale, where code 1 reflects ‘no necrosis’ and code 4 reflects ‘moderate necrosis’. For each co-formulation vehicle, a total of 5 skin samples were performed. However, due to variation in slicing the subcutaneous tissue for successful evaluation of the necrosis, not all injection sites could be assigned a score:
1, no necrosis
2, minimal necrosis
3, mild necrosis
4, moderate necrosis
   Isotonic co-formulation vehicle preparations containing 10% w/v to 20% w/v HP-B-CD were evaluated for the level of subcutaneous necrosis that they elicited upon subcutaneous injection. The results are presented in table 20.

TABLE 20
Necrosis scores 6 days post injections of subcutaneous tissue
necrosis for co-formulation necrosis for co-formulation vehicles
varying in percentage HP-B-CD

| | | | Number of successfully |
|---|---|---|---|
| Co-formulation | HP-B-CD | Necrosis, | scored skin samples of |
| vehicle | concentration | subcutane | total |
| 1 | 10% w/V | 2, 2, 2, 2, 3 | 5 of 5 |
| 2 | 12.5% w/V | 3, 3 | 2 of 5 |
| 3 | 15% w/V | 2, 2, 3 | 3 of 5 |
| 4 | 17.5% w/V | 2, 2, 3 | 3 of 5 |
| 5 | 20% w/V | 2, 3, 3, 4 | 4 of 5 |

### Concluding Remarks

A correlation was observed between increased HP-B-CD concentration in the co-formulation vehicle and necrosis at the injection site. In one case, the co-formulation vehicle containing 20% w/v HP-B-CD gave rise to moderate subcutaneous necrosis at the injection site. Formulations containing less than 20% w/v HP-B-CD all gave rise to only mild or minimal subcutaneous necrosis at the injection site. All co-formulation vehicles containing 10-20% w/v HP-B-CD were tolerated to an acceptable degree, those containing 10-17.5% w/v HP-B-CD being preferred.

### Example 12: Effect of Different Tonicity Agents on Subcutaneous Tolerance Upon Subcutaneous Injection

This example shows the effect on local tolerance of any one of three different tonicity agents (sorbitol, mannitol and trehalose) in otherwise identical isotonic co-formulation vehicles.

### Composition

The compositions of the tested co-formulation vehicles are shown in table 21.

TABLE 21
Composition of isotonic co-formulation vehicles prepared with
difference tonicity agents

| | Co-formulation vehicle | | |
|---|---|---|---|
| Ingredient | 6 | 7 | 8 |
| HP-B-CD | 15 | 15 | 15 |
| KLEPTOSE® (Roquette) | | | |
| (% w/V) | | | |
| (Average MS: 0.62, MS range: | | | |
| 0.58-0.68)¹ | | | |
| L-Histidine (mM) | 6 | 6 | 6 |
| Polysorbate 20 (mg/ml) | 0.1 | 0.1 | 0.1 |
| Sorbitol (mg/ml) | 24 | — | — |
| Mannitol (mg/ml) | — | 25 | — |
| Trehalose (mg/ml) | — | — | 46 |
| HCl | q.s. | q.s. | q.s. |
| NaOH | q.s. | q.s. | q.s. |
| WFl | To make | To make | To make |
| | 1 ml | 1 ml | 1 ml |
| pH | 6.0 | 6.0 | 6.0 |

¹MS: molar substitution, corresponds to hydroxypropyls per glucose unit

### Preparation Process

Formulations were prepared as described in example 1 except that addition of active pharmaceutical ingredients was abstained from.

### Method

The local tolerance from subcutaneous administration of isotonic vehicle preparations containing HP-B-CD and three different tonicity agents was studied in 2 live Landrace×YorkshirexDuroc (LYD) pigs by evaluation of the skin reactions resulting from subcutaneous administration of 600 μl vehicle preparation. The preparations were injected using syringes equipped with 25 G sized needles and 5 mm stoppers. Approx. 24 hours post injection necropsy was performed and skin samples sized 2×2 cm were fixed in neutral buffered formalin and trimmed into 4 μm sections using multi-knife, embedded in paraffin and subsequently HE stained. For the two samples, the severity of the subcutaneous tissue necrosis, inflammatory cell infiltration and haemorrhage distribution was assessed by a trained toxicopathologist using a light microscope and scored on a numerical scale, where code 1 reflects ‘no abnormality’ and code 3 reflects ‘mild severity’:
1, no abnormality
2, minimal severity
3, mild severity

TABLE 22
Severity scores 24 hours post subcutaneous injections of
subcutaneous tissue necrosis, inflammatory cell infiltration
and haemorrhage distribution for co-formulation vehicles
containing three types of tonicity agents

| Co- | | | | |
|---|---|---|---|---|
| formulation | Tonicity | | Inflammatory | Haemorrhage |
| vehicle | agent | Necrosis | cell infiltration | distribution |
| 6 | Sorbitol | 2, 1 | 2, 1 | 1, 1 |
| 7 | Mannitol | 2, 2 | 2, 3 | 1, 2 |
| 8 | Trehalose | 1, 1 | 2, 2 | 1, 2 |

### Concluding Remarks

The data presented in table 22 show that, overall, sorbitol was the tonicity agent that resulted in the least severe necrosis, inflammatory cell infiltration and haemorrhage. These observations confirm that sorbitol is the preferred tonicity agent for obtaining good and acceptable subcutaneous tolerability of the co-formulation containing the active pharmaceutical ingredients.

### Example 13: Confirmation on the Effect of Tonicity Agent Type in the Histidine-Buffered Formulation and the Effect of the Citrate-Buffered Formulation on the Subcutaneous Tolerance Upon Subcutaneous Injection

This experiment examines:
(1) the effect that the type of tonicity agent has on the local tolerance profile, upon subcutaneous injection, of an otherwise identical, histidine-buffered co-formulation; and
(2) the effect on the local tolerance profile, upon subcutaneous injection, of a co-formulation vehicle with a citrate-buffered formulation and no tonicity agent.

### Composition

The compositions of the evaluated co-formulations are described in table 23a and 23b.

TABLE 23a
Composition of isotonic histidine-buffered co-formulations

| | Co-formulation | |
|---|---|---|
| Ingredient | 19 | 20 |
| Cagrilintide drug substance (mg/ml) | 3.2 | 3.2 |
| Semaglutide drug substance (mg/ml) | 3.2 | 3.2 |
| HP-B-CD | 15 | 15 |
| KLEPTOSE® (Roquette) (% w/V) | | |
| (Average MS: 0.62, MS range: 0.58-0.68)¹ | | |
| L-Histidine (mM) | 6 | 6 |
| Trehalose (mg/ml) | 38.6 | — |
| Sorbitol (mg/ml) | — | 20 |
| Polysorbate 80 (mg/ml) | 0.05 | 0.05 |
| HCl | q.s. | q.s. |
| NaOH | q.s. | q.s. |
| WFl | To 1 ml | To 1 ml |
| pH | 6.0 | 6.0 |

¹MS: molar substitution, corresponds to hydroxypropyls per glucose unit

TABLE 23b
Composition of isotonic citrate-buffered co-formulation vehicle

| | Co-formulation vehicle |
|---|---|
| Ingredient | 21 |
| HP-B-CD | 25 |
| KLEPTOSE® (Roquette) (% w/V) | |
| (Average MS: 0.92, MS range: | |
| 0.81-0.99)¹ | |
| Citrate, 1 H₂O (mM) | 3 |
| HCl | q.s. |
| NaOH | q.s. |
| WFl | To 1 ml |
| pH | 5.7 |

¹MS: molar substitution, corresponds to hydroxypropyls per glucose unit

### Preparation Process

Co-formulations 19 and 21 were prepared as described in example 1. Coformulation vehicle 21 was prepared as described in example 1 except that addition of active pharmaceutical ingredients was abstained from.

### Method

The local tolerance upon subcutaneous administration of the co-formulations described in table 23a and 23b was studied in 8 live minipigs, by evaluation of the resulting skin lesions 6 days (necropsy) after subcutaneous administration of sample sizes of 750 μl using syringes equipped with 25 G sized needles and 5 mm stoppers. Skin samples sized 2×2 cm were collected at necropsy, fixed in neutral buffered formalin, trimmed using multi-knife, embedded in paraffin, cut in 4 μm thin sections, mounted to glass slides and subsequently hematoxylin-eosin (HE) stained. For the samples, the severity of the subcutaneous tissue necrosis was assessed by a trained toxicopathologist using a light microscope and scored on a numerical scale, where code 1 reflects ‘no abnormalities’ and code 5 reflects ‘marked severity’:
1, no abnormalities
2, minimal severity
3, mild severity
4, moderate severity
5, marked severity
   Results on scores of necrosis are shown in table 24a and table 24b.

TABLE 24a
Severity scores 6 days post injections of subcutaneous tissue
necrosis for histidine-buffered co-formulations

| | | | Necrosis scores for |
|---|---|---|---|
| Co-formulation | Buffer | Tonicity agent | four skin samples |
| 19 | Histidine | Trehalose | 1, 2, 3, 3 |
| 20 | Histidine | Sorbitol | 2, 2, 2, 2 |

TABLE 24b
Severity scores 6 days post injections of subcutaneous tissue
necrosis for citrate-buffered co-formulation vehicles

| Co-formulation | | | Necrosis scores for |
|---|---|---|---|
| vehicle | Buffer | Tonicity agent | eight skin samples |
| 21 | Citrate | None | 3, 4, 4, 4, 4, 5, 5, 5 |

### Concluding Remarks

The results presented in table 24a show that the type of tonicity agent included in the formulation affects its in vivo local tolerability. There is a corelation between tonicity agent and observed subcutaneous necrosis at the injection site. The subcutaneous injection of co-formulation 19, comprising trehalose, resulted in two events of mild necrosis (a score of 3). The subcutaneous injection of co-formulation 20, comprising sorbitol, resulted in minimal necrosis only (a score of 2), which is a better outcome. These results confirm that, in the case of this otherwise identical co-formulation vehicle, the co-formulation comprising 15% w/v HP-B-CD (Average MS: 0.62) and sorbitol is better than that comprising 15% w/v HP—B-CD (Average MS: 0.62) and trehalose.

The results presented in table 24b show that three events of marked necrosis (a score of 5) were observed with co-formulation 21 vehicle comprising 25% w/v HP-B-CD (Average MS: 0.92), citrate and no tonicity agent, confirming the unsuitability of this particular co-formulation for subcutaneous administration.

### Example 14: Effect of Hydroxypropyl-Substituted Cyclodextrins of Varying Type on Cagrilintide and Semaglutide Co-Formulation Physical and Chemical Stability

This example shows the effect of hydroxypropyl-alpha-cyclodextrin (HP-A-CD), hydroxypropyl-beta-cyclodextrin (HP-B-CD) and hydroxypropyl-gamma-cyclodextrin (HP-G-CD) on the formation of sub-visible particles and chemical degradation of cagrilintide in an otherwise identical cagrilintide and semaglutide co-formulation.

### Composition

The compositions of co-formulation 22, 23 and 24 are shown in table 25.

TABLE 25
Composition of co-formulation 22, 23 and 24

| | Co-formulation | | |
|---|---|---|---|
| Ingredient | 22 | 23 | 24 |
| Cagrilintide drug substance | 3.2 | 3.2 | 3.2 |
| (mg/ml) | | | |
| Semaglutide drug substance | 3.2 | 3.2 | 3.2 |
| (mg/ml) | | | |
| HP-A-CD | 15 | — | — |
| (CycloLab) (% w/v) | | | |
| (Average MS: 0.8, MS | | | |
| range: 0.5-0.9)¹ | | | |
| HP-B-CD KLEPTOSE ® | — | 15 | — |
| (Roquette) (% w/v) | | | |
| (Average MS: 0.62, MS | | | |
| range: 0.58-0.68)¹ | | | |
| HP-G-CD | — | — | 15 |
| (CycloLab) (% w/v) | | | |
| (Average MS: 0.6, MS | | | |
| range: 0.4-0.7)¹ | | | |
| L-Histidine (mM) | 6 | 6 | 6 |
| Sorbitol (mg/ml) | 20 | 20 | 20 |
| Polysorbate 80 (mg/ml) | 0.05 | 0.05 | 0.05 |
| HCl | q.s. | q.s. | q.s. |
| NaOH | q.s. | q.s. | q.s. |
| WFI | To make 1 ml | To make 1 ml | To make 1 ml |
| pH | 5.8 | 5.8 | 5.8 |

¹MS: molar substitution, corresponds to hydroxypropyls per glucose unit

### Preparation Process

Formulations were prepared as described in example 1.

### Methods

Samples used to determine sub-visible particle count were stored at stressed conditions, defined as:
Duration: 42 days
Temperature: 30° C.±2° C.
Stress condition: During storage, samples were inverted 360° to simulate patient use out of refrigerated storage. The rotations were performed 20 times three days every week, and 40 times two days every week.

The number of sub-visible particles was determined as described in example 4.

Samples used to determine the purity of cagrilintide were stored at 37° C. for up to 42 days. Purity of cagrilintide was determined using the following reversed phase high performance liquid chromatography (RP-HPLC) where the samples were analysed using a Kinetex C18, 2.6 μm column (4.6×150 mm) with a gradient elution of eluent A consisting of 90% v/v 0.09 M phosphate solution, pH 3.6 and 10% v/v acetonitrile, and eluent B consisting of 60% v/v acetonitrile and 20% v/v isopropanol. Chromatography was conducted with UV detection (210 nm) at 30° C. using a 10-100 μl injection volume and a flow rate of 0.7 ml/min. Purity of cagrilintide was quantified as being the area of the main peak divided by the area of all related peaks ×100%.

The same method was used to determine the purity of semaglutide in other experiments.

TABLE 26
Physical stability of cagrilintide and semaglutide
co-formulations formulated with hydroxypropyl
cyclodextrins of varying type

| | Type of | | | | | | |
|---|---|---|---|---|---|---|---|
| | hydroxy- | | | | | | |
| | propyl | Number of sub-visible particles | | | | | |
| Co- | substituted | as a function of time (days) | | | | | |
| formulation | cyclodextrin | 0 | 14 | 21 | 28 | 35 | 42 |

Particle size >5 μm

| | | | | | | | |
|---|---|---|---|---|---|---|---|
| 22 | HP-A-CD | 65 | 196 | 270 | 441 | 523 | 690 |
| 23 | HP-B-CD | 49 | 122 | 61 | 245 | 324 | 503 |
| 24 | HP-G-CD¹ | 453537 | — | — | — | — | — |

Particle size >10 μm

| | | | | | | | |
|---|---|---|---|---|---|---|---|
| 22 | HP-A-CD | 4 | 7 | 14 | 27 | 47 | 41 |
| 23 | HP-B-CD | 4 | 8 | 5 | 20 | 20 | 116 |
| 24 | HP-G-CD¹ | 107013 | — | — | — | — | — |

Particle size >25 μm

| | | | | | | | |
|---|---|---|---|---|---|---|---|
| 22 | HP-A-CD | 0 | 0 | 0 | 1 | 3 | 0 |
| 23 | HP-B-CD | 0 | 1 | 1 | 1 | 4 | 15 |
| 24 | HP-G-CD¹ | 316 | — | — | — | — | — |

Results for number of sub-visible particles are the mean of 3 replicates and has been rounded to nearest integer value
(—) Sampling not performed
¹For co-formulation 24 with HP-G-CD, sampling was discontinued earlier than for the other formulations because of rapid increases in particle counts.

TABLE 27
Chemical purity (%) of cagrilintide in cagrilintide and semaglutide co-
formulations formulated with hydroxypropyl cyclodextrins of varying
type

| | Type of | Cagrilintide purity (%) as a function | | | |
|---|---|---|---|---|---|
| Co- | hydroxypropyl | of time (days) at 37° C. | | | |
| formulation | cyclodextrin | 0 | 14 | 28 | 42 |
| 22 | HP-A-CD | 94.0% | 87.2% | 79.0% | 71.1% |
| 23 | HP-B-CD | 96.8% | 88.9% | 81.9% | 75.0% |

### Concluding Remarks

The results presented in table 26 show that in co-formulation 24 (HP-G-CD), high numbers of sub-visible particles were observed already at time zero, which preclude the use of HP-G-CD to co-formulate cagrilintide and semaglutide. The sampling for the analysis of sub-visible particle counts was discontinued for co-formulation 24 containing HP-G-CD after the initial analysis at time zero. For co-formulation 22 (HP-A-CD) and co-formulation 23 (HP—B-CD) virtually no increase in the number of sub-visible particles was observed.

The results presented in table 27 for the chemical purity of cagrilintide with either HP-A-CD or HP-B-CD, show a slightly more rapid decrease in cagrilintide purity in co-formulation 22, containing HP-A-CD, than in co-formulation 23 containing HP-B-CD. Based on results in table 26, either HP-A-CD or HP-B-CD is acceptable for co-formulations of cagrilintide and semaglutide. However, based on results in table 27, HP—B—CD is preferred compared to HP-A-CD for a cagrilintide and semaglutide co-formulation, due to the superior purity of cagrilintide when formulated with HP-B-CD.

### Example 15: Effect of the Molar Substitutiondegree of Hp-B-Cd on the Physical and Chemical Stability of Cagrilintide and Semaglutide Co-Formulations

This example shows the effect of the molar substitution of HP-B-CD on the formation of sub-visible particles, HMWP level and chemical purity of semaglutide in otherwise identical, citrate-buffered cagrilintide and semaglutide co-formulations.

### Composition

The compositions of co-formulations 25 to 32 are shown in table 28

TABLE 28
Compositions of citrate-buffered cagrilintide and semaglutide
co-formulations containing HP-B-CD excipients of
varying hydroxypropyl molar substitution degree

| | Co-formulation | | | | | | | |
|---|---|---|---|---|---|---|---|---|
| Ingredient | 25 | 26 | 27 | 28 | 29 | 30 | 31 | 32 |
| Cagrilintide drug | 3.2 | 3.2 | 3.2 | 3.2 | 3.2 | 3.2 | 3.2 | 3.2 |
| substance (mg/ml) | | | | | | | | |
| Semaglutide drug | 3.2 | 3.2 | 3.2 | 3.2 | 3.2 | 3.2 | 3.2 | 3.2 |
| substance (mg/ml) | | | | | | | | |
| HP-B-CD | 15 | — | 25 | — | — | — | — | — |
| KLEPTOSE ® | | | | | | | | |
| (Roquette) (% w/v) | | | | | | | | |
| (Average MS: 0.62, | | | | | | | | |
| MS range: 0.58-0.68)¹ | | | | | | | | |
| HP-B-CD | — | — | — | 25 | — | — | — | — |
| (CycloLab) (% w/v) | | | | | | | | |
| (Average MS: 0.67, | | | | | | | | |
| MS range: 0.6-0.9)¹ | | | | | | | | |
| HP-B-CD | — | — | — | — | 25 | — | — | — |
| Cavitron ® (Ashland) | | | | | | | | |
| (% w/v) | | | | | | | | |
| (Average MS: 0.68, | | | | | | | | |
| MS range: 0.58-0.72)¹ | | | | | | | | |
| HP-B-CD Trappsol ® | — | — | — | — | — | 25 | — | — |
| (CTD, Inc.) (% w/v) | | | | | | | | |
| (Average MS: 0.84, | | | | | | | | |
| MS range: 0.8-1.0) ¹ | | | | | | | | |
| HP-B-CD | — | 15 | — | — | — | — | 25 | — |
| KLEPTOSE ® | | | | | | | | |
| (Roquette) (% w/v) | | | | | | | | |
| (Average MS: 0.92, | | | | | | | | |
| MS range: 0.81-0.99)¹ | | | | | | | | |
| HP-B-CD Cavitron ® | — | — | — | — | — | — | — | 25 |
| (Ashland) (% w/v) | | | | | | | | |
| (Average MS: 1.08, | | | | | | | | |
| MS range: 0.86-1.14)¹ | | | | | | | | |
| Citrate, 1 H₂O (mM) | 3 | 3 | 3 | 3 | 3 | 3 | 3 | 3 |
| HCl | q.s. | q.s. | q.s. | q.s. | q.s. | q.s. | q.s. | q.s. |
| NaOH | q.s. | q.s. | q.s. | q.s. | q.s. | q.s. | q.s. | q.s. |
| WFI | To | To | To | To | To | To | To | To |
| | make | make | make | make | make | make | make | make |
| | 1 ml | 1 ml | 1 ml | 1 ml | 1 ml | 1 ml | 1 ml | 1 ml |
| pH | 5.8 | 5.8 | 5.8 | 5.8 | 5.8 | 5.8 | 5.8 | 5.8 |

¹MS: molar substitution, corresponds to hydroxypropyls per glucose unit

### Preparation Process

Formulations were prepared as described in example 1.

### Methods

Samples used to determine sub-visible particle count were stored at stressed conditions, defined as:
Duration: 28 days
Temperature: 30° C.±2° C.
Stress condition: During storage, samples were inverted 360° to simulate patient use out of refrigerated storage. The rotations were performed 20 times three days every week, and 40 times two days every week.
   The number of sub-visibles was quantified as described in example 4.

Samples used to determine the purity of semaglutide and HMWP levels were stored at 37° C. for up to 28 days. Purity of semaglutide was determined as in Example 14.

The level of covalently bound HMWP was determined using size exclusion chromatography (SEC). Samples were analysed using a Waters SEC 1.7 μm column (4.6×150 mm) with an isocratic elution consisting of 185 mM sodium chloride, 5 mM sodium dihydrogen phosphate monohydrate, 3 mM ortho-phosphate and 47% (v/v) isopropanol. Chromatography was conducted with UV detection (215 nm) at 50° C. using a 1-8 μl injection volume and a flow rate of 0.3 ml/min. HMWP was quantified as being the area of all components eluting before the main peak divided by the area of the main peak ×100%.

TABLE 29
Levels of sub-visible particles in citrate-buffered cagrilintide and
semaglutide co-formulations containing HP-B-CD excipients of
varying hydroxypropyl molar substitution degree

| | HP-B-CD | Number of sub-visible particles | | | |
|---|---|---|---|---|---|
| | average molar | as a function of time (days) | | | |
| Co-formulation | substitution¹ | 0 | 14 | 21 | 28 |

Particle size >5 μm

| | | | | | |
|---|---|---|---|---|---|
| 25 | 0.62 | 82 | 342 | 1232 | 25324 |
| 26 | 0.92 | 89 | 10108 | 13381 | 49863 |
| 27 | 0.62 | 126 | 550 | 370 | 448 |
| 28 | 0.67 | 59 | 356 | 191 | 887 |
| 29 | 0.68 | 77 | 27 | 629 | 4323 |
| 30 | 0.84 | 97 | 374 | 381 | 7031 |
| 31 | 0.92 | 106 | 244 | 761 | 3161 |
| 32 | 1.08 | 95 | 32626 | 54994 | 114586 |

Particle size >10 μm

| | | | | | |
|---|---|---|---|---|---|
| 25 | 0.62 | 11 | 73 | 349 | 4799 |
| 26 | 0.92 | 9 | 4007 | 5128 | 20604 |
| 27 | 0.62 | 13 | 175 | 93 | 131 |
| 28 | 0.67 | 13 | 103 | 53 | 262 |
| 29 | 0.68 | 6 | 10 | 131 | 1542 |
| 30 | 0.84 | 18 | 129 | 135 | 2224 |
| 31 | 0.92 | 32 | 59 | 271 | 765 |
| 32 | 1.08 | 6 | 13421 | 29840 | 59104 |

Particle size >25 μm

| | | | | | |
|---|---|---|---|---|---|
| 25 | 0.62 | 0 | 3 | 39 | 330 |
| 26 | 0.92 | 1 | 627 | 830 | 3461 |
| 27 | 0.62 | 0 | 3 | 8 | 13 |
| 28 | 0.67 | 0 | 1 | 6 | 27 |
| 29 | 0.68 | 0 | 1 | 5 | 245 |
| 30 | 0.84 | 1 | 3 | 34 | 296 |
| 31 | 0.92 | 3 | 4 | 46 | 56 |
| 32 | 1.08 | 1 | 825 | 7895 | 15815 |

Results for number of sub-visible particles are the mean of 3 replicates and has been rounded to nearest integer value
¹MS: molar substitution, corresponds to hydroxypropyls per glucose unit

TABLE 30
Levels of HMWP in citrate-buffered cagrilintide and semaglutide co-
formulations containing HP-B-CD excipients of varying hydroxypropyl
molar substitution degree

| | | Levels of HMWP (%) as a | | | |
|---|---|---|---|---|---|
| Co- | HP-B-CD molar | function of time (days) at 37° C. | | | |
| formulation | substitution¹ | 0 | 14 | 21 | 28 |
| 25 | 0.62 | 0.03% | 0.08% | 0.13% | 0.18% |
| 26 | 0.92 | 0.05% | 0.12% | 0.18% | 0.22% |
| 27 | 0.62 | 0.04% | 0.08% | 0.14% | 0.15% |
| 28 | 0.67 | 0.05% | 0.11% | 0.17% | 0.20% |
| 29 | 0.68 | 0.04% | 0.09% | 0.16% | 0.20% |
| 30 | 0.84 | 0.05% | 0.12% | 0.17% | 0.21% |
| 31 | 0.92 | 0.04% | 0.13% | 0.22% | 0.27% |
| 32 | 1.08 | 0.11% | 0.47% | 0.61% | 0.76% |

¹MS: molar substitution, corresponds to hydroxypropyls per glucose unit

TABLE 31
Chemical purity (%) of semaglutide in citrate-buffered cagrilintide and
semaglutide co-formulations containing HP-B-CD excipients of
varying hydroxypropyl molar substitution degree

| | | Semaglutide purity (%) as a | | | |
|---|---|---|---|---|---|
| Co- | HP-B-CD molar | function of time (days) at 37° C. | | | |
| formulation | substitution¹ | 0 | 14 | 21 | 28 |
| 25 | 0.62 | 97.0% | 95.3% | 94.4% | 93.8% |
| 26 | 0.92 | 97.0% | 95.1% | 93.9% | 93.4% |
| 27 | 0.62 | 97.0% | 95.2% | 94.3% | 93.4% |
| 28 | 0.67 | 96.9% | 95.0% | 94.1% | 93.5% |
| 29 | 0.68 | 97.0% | 95.3% | 94.4% | 93.5% |
| 30 | 0.84 | 97.1% | 95.4% | 94.6% | 93.8% |
| 31 | 0.92 | 96.9% | 94.4% | 93.4% | 92.3% |
| 32 | 1.08 | 96.6% | 91.4% | 89.1% | 87.5% |

¹MS: molar substitution, corresponds to hydroxypropyls per glucose unit

### Concluding Remarks

The results presented in tables 29, 30, and 31 show that the physical stability, the formation of HMWP, and the chemical purity of semaglutide is dependent on the molar substitution of the HP-B-CD when studied in a citrate-buffered cagrilintide and semaglutide co-formulation. After 28 days, HMWP levels were lowest and there was virtually no increase in the particle count in co-formulation 27, containing 25% w/v HP-B-CD (Average MS: 0.62). In contrast, in co-formulation 32 containing 25% w/v HP-B-CD (Average MS: 1.08), a large increase in sub-visible particle count was observed after only 14 days and it was also in this formulation that the highest levels of HMWP were observed after 28 days.

All citrate-buffered cagrilintide and semaglutide co-formulations containing 25% w/V HP-B-CD (Average MS: 0.92 or less), were physically and chemically stable, those containing 25% w/v HP-B-CD having an average MS of 0.68 or less being the most stable.

After only 14 days, an increase in the sub-visible particle count was seen in co-formulation 26, containing 15% w/v HP-B-CD (Average MS: 0.92), indicating the physical instability of this particular co-formulation.

Co-formulation 25, containing 15% w/v HP-B-CD (Average MS: 0.62), showed acceptable chemical and physical stability.

However, the histidine-buffered cagrilintide and semaglutide co-formulations 33 to 37, containing 15% w/v HP-B-CD, are preferred due to their superior physical stability. With histidine as buffer and sorbitol as tonicity agent, the preferred HP-B-CD molar substitution range was widened to an average of 0.62-0.92 (or a total of 0.58-1.0).

### Example 16: Effect of Molar Substitutiondegree of Hp-B-Cd on Physical Stability of Cagrilintide and Semaglutide Co-Formulations

This example shows the effect of the molar substitution degree of HP-B-CD on the levels of sub-visible particles in otherwise identical, histidine-buffered cagrilintide and semaglutide co-formulations.

### Composition

The compositions of co-formulation 33 to 38 are shown in table 32.

TABLE 32
Compositions of histidine-buffered cagrilintide and semaglutide co-
formulation 33 to 38 containing HP-B-CD excipients of varying
hydroxypropyl molar substitution degree

| | Co-formulation | | | | | |
|---|---|---|---|---|---|---|
| Ingredient | 33 | 34 | 35 | 36 | 37 | 38 |
| Cagrilintide drug substance | 3.2 | 3.2 | 3.2 | 3.2 | 3.2 | 3.2 |
| (mg/ml) | | | | | | |
| Semaglutide drug substance | 3.2 | 3.2 | 3.2 | 3.2 | 3.2 | 3.2 |
| (mg/ml) | | | | | | |
| HP-B-CD | 15 | — | — | — | — | — |
| KLEPTOSE ® (Roquette) | | | | | | |
| (% w/v) | | | | | | |
| (Average MS: 0.62, MS range: | | | | | | |
| 0.58-0.68)¹ | | | | | | |
| HP-B-CD | — | 15 | — | — | — | — |
| (CycloLab) (% w/v) (Average | | | | | | |
| MS: 0.67, MS range: 0.6-0.9)¹ | | | | | | |
| HP-B-CD | — | — | 15 | — | — | — |
| Cavitron ® (Ashland) (% w/v) | | | | | | |
| (Average MS: 0.68, MS range: | | | | | | |
| 0.58-0.72)¹ | | | | | | |
| HP-B-CD Trappsol ® | — | — | — | 15 | — | — |
| (CTD, Inc.) (% w/v) | | | | | | |
| (Average MS: 0.84, MS | | | | | | |
| range: 0.8-1.0) ¹ | | | | | | |
| HP-B-CD | — | — | — | — | 15 | — |
| KLEPTOSE ® (Roquette) (% | | | | | | |
| w/v) | | | | | | |
| (Average MS: 0.92, MS range: | | | | | | |
| 0.81-0.99)¹ | | | | | | |
| HP-B-CD Cavitron ® (Ashland) | — | — | — | — | — | 15 |
| (% w/v) (Average MS: 1.08, | | | | | | |
| MS range: 0.86-1.14)¹ | | | | | | |
| L-Histidine (mM) | 6 | 6 | 6 | 6 | 6 | 6 |
| Sorbitol (mg/ml) | 20 | 20 | 20 | 20 | 20 | 20 |
| Polysorbate 80 (mg/ml) | 0.05 | 0.05 | 0.05 | 0.05 | 0.05 | 0.05 |
| HCl | q.s. | q.s. | q.s. | q.s. | q.s. | q.s. |
| NaOH | q.s. | q.s. | q.s. | q.s. | q.s. | q.s. |
| WFI | To | To | To | To | To | To |
| | make 1 | make 1 | make 1 | make 1 | make 1 | make 1 |
| | ml | ml | ml | ml | ml | ml |
| pH | 5.8 | 5.8 | 5.8 | 5.8 | 5.8 | 5.8 |

¹MS: molar substitution, corresponds to hydroxypropyls per glucose unit

### Preparation Process

Formulations were prepared as described in example 1.

### Methods

Samples used to determine the number of sub-visible particles were stored at stressed conditions, defined as:
Duration: 28 days
Temperature: 30° C.±2° C.
Stress condition: During storage, samples were inverted 360° to simulate patient use out of refrigerated storage. The rotations were performed 20 times three days every week, and 40 times two days every week.

The number of sub-visible particles was quantified as described in example 4.

TABLE 33
Number of sub-visible particles in histidine-buffered cagrilintide and
semaglutide co-formulations containing HP-B-CD of varying
hydroxypropyl molar substitution degree

| | | Number of sub-visible particles | | | |
|---|---|---|---|---|---|
| Co- | HP-B-CD molar | as a function of time (days) | | | |
| formulation | substitution¹ | 0 | 14 | 21 | 28 |

Particle size >5 μm

| | | | | | |
|---|---|---|---|---|---|
| 33 | 0.62 | 19 | 300 | 252 | 460 |
| 34 | 0.67 | 37 | 176 | 220 | 66 |
| 35 | 0.68 | 22 | 175 | 453 | 290 |
| 36 | 0.84 | 17 | 270 | 221 | 112 |
| 37 | 0.92 | 51 | 873 | 804 | 473 |
| 38 | 1.08 | 71 | 1311 | 1765 | 1621 |

Particle size >10 μm

| | | | | | |
|---|---|---|---|---|---|
| 33 | 0.62 | 1 | 14 | 6 | 61 |
| 34 | 0.67 | 6 | 9 | 6 | 4 |
| 35 | 0.68 | 1 | 5 | 23 | 56 |
| 36 | 0.84 | 1 | 13 | 0 | 6 |
| 37 | 0.92 | 3 | 45 | 24 | 117 |
| 38 | 1.08 | 4 | 143 | 416 | 579 |

Particle size >25 μm

| | | | | | |
|---|---|---|---|---|---|
| 33 | 0.62 | 0 | 0 | 0 | 3 |
| 34 | 0.67 | 0 | 0 | 0 | 0 |
| 35 | 0.68 | 0 | 3 | 0 | 1 |
| 36 | 0.84 | 0 | 0 | 0 | 4 |
| 37 | 0.92 | 0 | 1 | 1 | 20 |
| 38 | 1.08 | 1 | 0 | 14 | 27 |

Results for number of sub-visible particles are the mean of 3 replicates and have been rounded to nearest integer value
¹MS: molar substitution, corresponds to hydroxypropyls per glucose unit

### Concluding Remarks

The results presented in table 33 show that the histidine-buffered co-formulations 33-37, comprising HP-B-CD with a wide range of molar substitutions (Average MS: 0.62-0.92), remained physically stable for 28 days.

In contrast, in co-formulation 38, comprising HP-B-CD (Average MS 1.08), was not physically stable after 14 days.

The results indicate a synergistic effect between the cyclodextrin and the other excipients in the co-formulation, widening the preferred range of molar substitution (Average MS 0.62-0.92).

### Example 17: Effect of Type of Beta-Cyclodextrin Substitution on the Physical Stability of the Co-Formulation

This example shows the effect of sulfobutylether-β-cyclodextrin (SBE-B-CD) and hydroxypropyl-beta-cyclodextrin on the physical stability of otherwise identical cagrilintide and semaglutide co-formulations.

### Composition

The compositions of co-formulations 39 and 40 are shown in table 36.

TABLE 36
Composition of co-formulation containing either
HP-B-CD or SBE-B-CD

| | Co-formulation | |
|---|---|---|
| Ingredient | 39 | 40 |
| Cagrilintide drug substance (mg/ml) | 3.2 | 3.2 |
| Semaglutide drug substance (mg/ml) | 3.2 | 3.2 |
| HP-B-CD | 15 | — |
| KLEPTOSE ® (Roquette) (% w/v) | | |
| (Average MS: 0.62, MS range: 0.58-0.68)¹ | | |
| SBE-B-CD | — | 15 |
| (CycloLab) (% w/v) | | |
| (Average MS: 0.87, MS range: 0.84-0.99)¹ | | |
| L-Histidine (mM) | 6 | 6 |
| Sorbitol (mg/ml) | 20 | 20 |
| Polysorbate 80 (mg/ml) | 0.05 | 0.05 |
| HCl | q.s. | q.s. |
| NaOH | q.s. | q.s. |
| WFI | To 1 ml | To 1 ml |
| pH | 5.8 | 5.8 |

¹MS: molar substitution, corresponds to sulfobutyl ethers/hydroxypropyls per glucose unit

### Preparation Process

Formulations were prepared as described in example 1.

### Methods

Samples used to determine the number of sub-visible particles were stored at stressed conditions, defined as:
Duration: 35 days
Temperature: 30° C.±2° C.
Stress condition: During storage, samples were inverted 360° to simulate patient use out of refrigerated storage. The rotations were performed 20 times three days every week, and 40 times two days every week.

The number of sub-visible particles was quantified as described in example 4.

TABLE 37
Levels of sub-visible particles in cagrilintide and semaglutide co-
formulations containing either HP-B-CD or SBE-B-CD

| | | Number of sub-visible particles | | | | |
|---|---|---|---|---|---|---|
| Co- | Type of | as a function of time (days) | | | | |
| formulation | substitution | 0 | 14 | 21 | 28 | 35 |

Particle size >5 μm

| | | | | | | |
|---|---|---|---|---|---|---|
| 39 | HP-B-CD | 49 | 122 | 61 | 245 | 324 |
| 40 | SBE-B-CD | 317 | 1686 | 6940 | 20714 | 280478 |

Particle size >10 μm

| | | | | | | |
|---|---|---|---|---|---|---|
| 39 | HP-B-CD | 4 | 8 | 5 | 20 | 20 |
| 40 | SBE-B-CD | 13 | 640 | 3503 | 10248 | 138324 |

Particle size >25 μm

| | | | | | | |
|---|---|---|---|---|---|---|
| 39 | HP-B-CD | 0 | 1 | 1 | 1 | 4 |
| 40 | SBE-B-CD | 0 | 126 | 738 | 2008 | 29834 |

Results for number of sub-visible particles are the mean of 3 replicates and has been rounded to nearest integer value

### Concluding Remarks

The results in table 37 show that when using SBE-B-CD to co-formulate cagrilintide and semaglutide, a large increase in the number of sub-visible particles is observed after 14 days; that is, the co-formulation is physically unstable. When using a HP-B-CD instead, virtually no increase is observed during the 35 days duration of the study; that is, the co-formulation is physically stable.

In contrast to what we have shown for hydroxypropyl-beta-cyclodextrin, these results demonstrate that sulfobutylether-β-cyclodextrin (SBE-B-CD) is not a suitable cyclodextrin to use for co-formulating cagrilintide and semaglutide.

### Example 18: Effect of pH on the Physical and Chemical Stability of the Co-Formulation

This example shows the effect of pH on the physical and chemical stability of cagrilintide in otherwise identical cagrilintide and semaglutide co-formulations.

### Composition

The compositions of co-formulation 41 to 45 are shown in table 38.

TABLE 38
Composition of co-formulation with varying pH

| | Co-formulation | | | | |
|---|---|---|---|---|---|
| Ingredient | 41 | 42 | 43 | 44 | 45 |
| Cagrilintide drug substance (mg/ml) | 3.2 | 3.2 | 3.2 | 3.2 | 3.2 |
| Semaglutide drug substance (mg/ml) | 3.2 | 3.2 | 3.2 | 3.2 | 3.2 |
| HP-B-CD | 15 | 15 | 15 | 15 | 15 |
| KLEPTOSE ® (Roquette) (% w/v) | | | | | |
| (Average MS: 0.62, | | | | | |
| MS range: 0.58-0.68)¹ | | | | | |
| L-Histidine (mM) | 6 | 6 | 6 | 6 | 6 |
| Sorbitol (mg/ml) | 20 | 20 | 20 | 20 | 20 |
| Polysorbate 80 (mg/ml) | 0.05 | 0.05 | 0.05 | 0.05 | 0.05 |
| HCl | q.s. | q.s. | q.s. | q.s. | q.s. |
| NaOH | q.s. | q.s. | q.s. | q.s. | q.s. |
| WFI (ml) | To 1 | To 1 | To 1 | To 1 | To 1 |
| pH | 5.5 | 5.6 | 5.7 | 5.8 | 6.0 |

¹MS: molar substitution, corresponds to hydroxypropyls per glucose unit

### Preparation Process

Formulations were prepared as described in example 1.

### Methods

Samples used to determine the number of sub-visible particles were stored at stressed conditions, defined as:
Duration: 28 days
Temperature: 30° C.±2° C.
Stress condition: During storage, samples were inverted 360° to simulate patient use out of refrigerated storage. The rotations were performed 20 times three days every week, and 40 times two days every week.

The number of sub-visible particles was quantified as described in example 4.

Samples used to determine the purity of cagrilintide was stored at 37° C. for up to 28 days. The purity of cagrilintide was determined as described in example 14.

TABLE 39
Physical stability of the cagrilintide and semaglutide co-formulations
with varying pH within the pH-range 5.5 to 6.0

| | | Number of sub-visible particles | | | |
|---|---|---|---|---|---|
| | | as a function of time (days) | | | |
| Co-formulation | pH | 0 | 14 | 21 | 28 |

Particle size >5 μm

| | | | | | |
|---|---|---|---|---|---|
| 41 | 5.5 | 70 | 6594 | — | 523259 |
| 42 | 5.6 | 69 | 174 | 730 | 1355 |
| 43 | 5.7 | 120 | 117 | 232 | 297 |
| 44 | 5.8 | 49 | 122 | 61 | 245 |
| 45 | 6.0 | 33 | 178 | 133 | 60 |

Particle size >10 μm

| | | | | | |
|---|---|---|---|---|---|
| 41 | 5.5 | 6 | 851 | — | 385284 |
| 42 | 5.6 | 6 | 9 | 200 | 476 |
| 43 | 5.7 | 3 | 5 | 8 | 18 |
| 44 | 5.8 | 4 | 8 | 5 | 20 |
| 45 | 6.0 | 4 | 6 | 3 | 9 |

Particle size >25 μm

| | | | | | |
|---|---|---|---|---|---|
| 41 | 5.5 | 1 | 4 | — | 190781 |
| 42 | 5.6 | 0 | 0 | 18 | 64 |
| 43 | 5.7 | 0 | 0 | 0 | 0 |
| 44 | 5.8 | 0 | 1 | 1 | 1 |
| 45 | 6.0 | 1 | 0 | 0 | 1 |

Results for number of sub-visible particles are the mean of 3 replicates and has been rounded to nearest integer value
(—) sampling not performed

TABLE 40
Chemical purity (%) of cagrilintide in the cagrilintide and semaglutide
co-formulations with varying pH within the pH-range 5.5 to 6.0

| | | Cagrilintide purity (%) as a | | |
|---|---|---|---|---|
| Co- | | function of time (days) at 37° C. | | |
| formulation | pH | 0 | 14 | 28 |
| 41 | 5.5 | 97.1% | 92.1% | 85.9% |
| 42 | 5.6 | 97.1% | 91.6% | 85.0% |
| 43 | 5.7 | 97.0% | 90.6% | 83.2% |
| 44 | 5.8 | 96.9% | 88.9% | 81.9% |
| 45 | 6.0 | 96.9% | 88.2% | 78.3% |

### Concluding Remarks

The results presented in tables 39 and 40 show that the physical and chemical stability of semaglutide and cagrilintide depend on the pH of the formulation: the highest pH results in the lowest cagrilintide purity after 28 days at 37° C.; the lowest pH results in an increase in the number of sub-visible particles after 14 days. Based on these physical and chemical stability results, the preferred pH range for this particular cagrilintide and semaglutide co-formulation is 5.6-6.0, whilst the pH of 5.5 did not result in a co-formulation of acceptable physical stability.

### Example 19: Effect of Cagrilintide and Semaglutide Concentration Ratios on the Physical Stability of the Co-Formulation

This example shows the effect of different concentration ratios of cagrilintide and semaglutide on the levels of sub-visible particles observed in the co-formulation.

### Composition

The composition of histidine-buffered co-formulations 46 to 50 are shown in table 41, and the composition of histidine-buffered co-formulations 51 to 61 are shown in table 42.

TABLE 41
Composition of histidine-buffered co-formulations with varying
cagrilintide and semaglutide concentration ratios

| | Co-formulation | | | | |
|---|---|---|---|---|---|
| Ingredient | 46 | 47 | 48 | 49 | 50 |
| Cagrilintide drug substance | 3.2 | 3.2 | 3.2 | 3.2 | 3.2 |
| (mg/ml) | | | | | |
| Semaglutide drug substance | 3.2 | 4.8 | 6.4 | 8.0 | 9.6 |
| (mg/ml) | | | | | |
| HP-B-CD KLEPTOSE ® | 15 | 15 | 15 | 15 | 15 |
| (Roquette) (% w/v) | | | | | |
| (Average MS: 0.62, MS | | | | | |
| range: 0.58-0.68)¹ | | | | | |
| L-Histidine (mM) | 6 | 6 | 6 | 6 | 6 |
| Sorbitol (mg/ml) | 20 | 20 | 20 | 20 | 20 |
| Polysorbate 80 (mg/ml) | 0.05 | 0.05 | 0.05 | 0.05 | 0.05 |
| HCl | q.s. | q.s. | q.s. | q.s. | q.s. |
| NaOH | q.s. | q.s. | q.s. | q.s. | q.s. |
| WFI | To 1 ml | To 1 ml | To 1 ml | To 1 ml | To 1 ml |
| pH | 5.8 | 5.8 | 5.8 | 5.8 | 5.8 |

¹MS: molar substitution, corresponds to hydroxypropyls per glucose unit

TABLE 42
Composition of histidine-buffered co-formulations with modified
composition with varying cagrilintide and semaglutide concentration ratios

| | Co-formulation | | | | | | | | | | |
|---|---|---|---|---|---|---|---|---|---|---|---|
| Ingredient | 51 | 52 | 53 | 54 | 55 | 56 | 57 | 58 | 59 | 60 | 61 |
| Cagrilintide drug | 3.2 | 3.2 | 3.2 | 3.2 | 3.2 | 3.2 | 3.2 | 3.2 | 3.2 | 3.2 | 3.2 |
| substance | | | | | | | | | | | |
| (mg/ml) | | | | | | | | | | | |
| Semaglutide | 3.2 | 4.8 | 6.4 | 8.0 | 9.6 | 9.6 | 9.6 | 9.6 | 10.7 | 12.8 | 16 |
| drug substance | | | | | | | | | | | |
| (mg/ml) | | | | | | | | | | | |
| HP-B-CD | 20 | 20 | 20 | 20 | 15 | 20 | 20 | 20 | 20 | 20 | 20 |
| KLEPTOSE ® | | | | | | | | | | | |
| (Roquette) (% | | | | | | | | | | | |
| w/v) | | | | | | | | | | | |
| (Average MS: | | | | | | | | | | | |
| 0.62, MS range: | | | | | | | | | | | |
| 0.58-0.68)¹ | | | | | | | | | | | |
| L-Histidine (mM) | 6 | 6 | 6 | 6 | 6 | 6 | 6 | 6 | 6 | 6 | 6 |
| Sorbitol (mg/ml) | 35 | 35 | 35 | 35 | 35 | 20 | 35 | 35 | 35 | 35 | 35 |
| Polysorbate 80 | 1.78 | 1.78 | 1.78 | 1.78 | 0.05 | 0.05 | 0.05 | 1.78 | 1.78 | 1.78 | 1.78 |
| (mg/ml) | | | | | | | | | | | |
| HCl | q.s. | q.s. | q.s. | q.s. | q.s. | q.s. | q.s. | q.s. | q.s. | q.s. | q.s. |
| NaOH | q.s. | q.s. | q.s. | q.s. | q.s. | q.s. | q.s. | q.s. | q.s. | q.s. | q.s. |
| WFI | To 1 | To 1 | To 1 | To 1 | To 1 | To 1 | To 1 | To 1 | To 1 | To 1 | To 1 |
| | ml | ml | ml | ml | ml | ml | ml | ml | ml | ml | ml |
| pH | 5.8 | 5.8 | 5.8 | 5.8 | 5.8 | 5.8 | 5.8 | 5.8 | 5.8 | 5.8 | 5.8 |

¹MS: molar substitution, corresponds to hydroxypropyls per glucose unit

### Preparation Process

Formulations were prepared as described in example 1.

Methods Samples used to determine the number of sub-visible particles were stored at stressed conditions, defined as:
Duration: 28 days
Temperature: 30° C.±2° C.
Stress condition: During storage, samples were inverted 360° to simulate patient use out of refrigerated storage. The rotations were performed 20 times three days every week, and 40 times two days every week.
   The number of sub-visible particles was quantified as described in example 4.

TABLE 43
Levels of sub-visible particles in the co-formulation containing
different concentration ratios of cagrilintide and semaglutide

| Co- | Cagrilintide/ | Number of sub-visible particles | | | |
|---|---|---|---|---|---|
| formu- | semaglutide | as a function of time (days) | | | |
| lation | concentration (ratio) | 0 | 14 | 21 | 28 |

Particle size >5 μm

| | | | | | |
|---|---|---|---|---|---|
| 46 | 3.2/3.2 mg/ml (1:1) | 207 | 214 | 542 | 341 |
| 47 | 3.2/4.8 mg/ml (1:1.5) | 65 | 133 | 214 | 591 |
| 48 | 3.2/6.4 mg/ml (1:2) | 55 | 76 | 198 | 134 |
| 49 | 3.2/8.0 mg/ml (1:2.5) | 44 | 86 | 262 | 139 |
| 50 | 3.2/9.6 mg/ml (1:3) | 56 | 184 | 137 | 540 |
| 51 | 3.2/3.2 mg/ml (1:1) | 297 | 467 | 650 | 11684 |
| 52 | 3.2/4.8 mg/ml (1:1.5) | 377 | 541 | 331 | 14453 |
| 53 | 3.2/6.4 mg/ml (1:2) | 588 | 501 | 980 | 45916 |
| 54 | 3.2/8.0 mg/ml (1:2.5) | 452 | 341 | 712 | 13108 |
| 55 | 3.2/9.6 mg/ml (1:3) | 37 | 226 | 121 | 161 |
| 56 | 3.2/9.6 mg/ml (1:3) | 55 | 77 | 290 | 315 |
| 57 | 3.2/9.6 mg/ml (1:3) | 41 | 181 | 273 | 368 |
| 58 | 3.2/9.6 mg/ml (1:3) | 312 | 457 | 445 | 25494 |
| 59 | 3.2/10.7 mg/ml (1:3.33) | 1240 | 594 | 1043 | 53301 |
| 60 | 3.2/12.8 mg/ml (1:4) | 1459 | 549 | 320 | 77697 |
| 61 | 3.2/16 mg/ml (1:5) | 1025 | 568 | 877 | 5202 |

Particle size >10 μm

| | | | | | |
|---|---|---|---|---|---|
| 46 | 3.2/3.2 mg/ml (1:1) | 18 | 31 | 25 | 51 |
| 47 | 3.2/4.8 mg/ml (1:1.5) | 5 | 9 | 22 | 106 |
| 48 | 3.2/6.4 mg/ml (1:2) | 3 | 9 | 18 | 24 |
| 49 | 3.2/8.0 mg/ml (1:2.5) | 10 | 9 | 13 | 13 |
| 50 | 3.2/9.6 mg/ml (1:3) | 1 | 9 | 8 | 130 |
| 51 | 3.2/3.2 mg/ml (1:1) | 38 | 45 | 79 | 1146 |
| 52 | 3.2/4.8 mg/ml (1:1.5) | 69 | 94 | 80 | 2053 |
| 53 | 3.2/6.4 mg/ml (1:2) | 74 | 81 | 171 | 7608 |
| 54 | 3.2/8.0 mg/ml (1:2.5) | 78 | 57 | 118 | 1651 |
| 55 | 3.2/9.6 mg/ml (1:3) | 5 | 9 | 5 | 14 |
| 56 | 3.2/9.6 mg/ml (1:3) | 4 | 8 | 22 | 59 |
| 57 | 3.2/9.6 mg/ml (1:3) | 0 | 15 | 27 | 61 |
| 58 | 3.2/9.6 mg/ml (1:3) | 50 | 79 | 74 | 3233 |
| 59 | 3.2/10.7 mg/ml (1:3.33) | 147 | 72 | 165 | 6793 |
| 60 | 3.2/12.8 mg/ml (1:4) | 155 | 84 | 51 | 10270 |
| 61 | 3.2/16 mg/ml (1:5) | 80 | 99 | 189 | 382 |

Particle size >25 μm

| | | | | | |
|---|---|---|---|---|---|
| 46 | 3.2/3.2 mg/ml (1:1) | 1 | 0 | 4 | 1 |
| 47 | 3.2/4.8 mg/ml (1:1.5) | 0 | 1 | 0 | 6 |
| 48 | 3.2/6.4 mg/ml (1:2) | 0 | 1 | 3 | 3 |
| 49 | 3.2/8.0 mg/ml (1:2.5) | 1 | 0 | 3 | 3 |
| 50 | 3.2/9.6 mg/ml (1:3) | 0 | 1 | 0 | 9 |
| 51 | 3.2/3.2 mg/ml (1:1) | 0 | 0 | 1 | 13 |
| 52 | 3.2/4.8 mg/ml (1:1.5) | 4 | 0 | 3 | 60 |
| 53 | 3.2/6.4 mg/ml (1:2) | 8 | 4 | 6 | 157 |
| 54 | 3.2/8.0 mg/ml (1:2.5) | 8 | 3 | 3 | 25 |
| 55 | 3.2/9.6 mg/ml (1:3) | 0 | 0 | 1 | 0 |
| 56 | 3.2/9.6 mg/ml (1:3) | 3 | 0 | 0 | 3 |
| 57 | 3.2/9.6 mg/ml (1:3) | 0 | 0 | 4 | 0 |
| 58 | 3.2/9.6 mg/ml (1:3) | 1 | 5 | 0 | 42 |
| 59 | 3.2/10.7 mg/ml (1:3.33) | 1 | 0 | 5 | 97 |
| 60 | 3.2/12.8 mg/ml (1:4) | 3 | 3 | 1 | 68 |
| 61 | 3.2/16 mg/ml (1:5) | 8 | 4 | 26 | 8 |

Results for number of sub-visible particles are the mean of 3 replicates and has been rounded to nearest integer value

### Concluding Remarks

The results presented in tables 43 show that after 21 days, virtually no increase in sub-visible particle count was seen in co-formulation 46-60 containing 3.2 mg/ml cagrilintide and up to 12 mg/ml semaglutide.

After 14, an increase in sub-visible particle count was seen for co-formulation 61 containing 3.2 mg/ml cagrilintide and 16 mg/ml semaglutide.

All histidine-buffered co-formulations 46 to 61 comprising 3.2 mg/ml cagrilintide and up to 16 mg/ml semaglutide was physically stable.

### Example 20: Effect of Concentration of Hp-B-Cd on the Physical Stability of the Co-Formulation

This example shows the effect of HP-B-CD concentration on the physical stability of the cagrilintide and semaglutide co-formulation, when the co-formulation is exposed to physical stress.

### Composition

The composition of co-formulation 62 to 65 with the histidine-buffered composition is shown in table 44.

TABLE 44
Composition of co-formulation with varying HP-B-CD concentrations

| | Co-formulation | | | |
|---|---|---|---|---|
| Ingredient | 62 | 63 | 64 | 65 |
| Cagrilintide drug substance (mg/ml) | 3.2 | 3.2 | 3.2 | 3.2 |
| Semaglutide drug substance (mg/ml) | 3.2 | 3.2 | 3.2 | 3.2 |
| HP-B-CD KLEPTOSE ® | 7.5 | 10 | 12.5 | 15 |
| (Roquette) (% w/v) | | | | |
| (Average MS: 0.62, MS | | | | |
| range: 0.58-0.68)¹ | | | | |
| L-Histidine (mM) | 6 | 6 | 6 | 6 |
| Sorbitol (mg/ml) | 20 | 20 | 20 | 20 |
| Polysorbate 80 (mg/ml) | 0.05 | 0.05 | 0.05 | 0.05 |
| HCl | q.s. | q.s. | q.s. | q.s. |
| NaOH | q.s. | q.s. | q.s. | q.s. |
| WFI | To 1 ml | To 1 ml | To 1 ml | To 1 ml |
| pH | 5.8 | 5.8 | 5.8 | 5.8 |

¹MS: molar substitution, corresponds to hydroxypropyls per glucose unit

### Preparation Process

Formulations were prepared as described in example 1.

### Method

The propensity of cagrilintide and semaglutide in the co-formulation to aggregate and form peptide fibrils was measured using a Thioflavin T (ThT) fluorescence stress assay as described in example 2.

TABLE 45
Physical stability for cagrilintide and semaglutide co-formulation with
varying HP-B-CD concentrations

| Co-formulation | HP-B-CD (MS: 0.62)² | Lag time until fibrillation |
|---|---|---|
| 62 | 7.5% w/v | 17.66 hours¹ |
| 63 | 10% w/v | 28.06 hours¹ |
| 64 | 12.5% w/v | 70.97 hours¹ |
| 65 | 15% w/v | 119.0 hours¹ |

¹Result is the mean of 6 replicates
²MS: molar substitution, corresponds to hydroxypropyls per glucose unit

### Concluding Remarks

The results presented in table 45 show that the physical stability of the cagrilintide and semaglutide co-formulation is dependent upon the concentration of HP-B-CD, with lower concentrations resulting in shorter lag time until fibrillation occurs. The co-formulation comprising 7.5% w/v HP-B-CD was the least stable. The co-formulation comprising 15% w/V HP-B-CD was the most stable.

### Example 21: Local Tolerance, in Pigs, of Subcutaneously Injected Vehicle Formulations Varying in Hp-B-Cd Content and Molar Substitutiondegree, as Well as in Overall Buffer Composition

This experiment examined:
(1) the effect that HP-B-CD concentration and average MS (0.62 versus 0.92) had on the local tolerance profile, upon subcutaneous injection.
(2) the effect that the formulation vehicle has on the local tolerance profile in the presence of HP-B-CD, upon subcutaneous injection.

### Composition

The compositions of the tested co-formulation vehicles are shown in table 46.

TABLE 46
Composition of co-formulation vehicles varying in HP-B-CD content
and molar substitution degree as well as in overall buffer composition

| | Co-formulation vehicles | | | | | | | |
|---|---|---|---|---|---|---|---|---|
| Ingredient | 9 | 10 | 11 | 12 | 13 | 14 | 15 | 16 |
| HP-B-CD | 15 | 20 | 22 | 22 | 25 | 20 | 22 | — |
| KLEPTOSE ® | | | | | | | | |
| (Roquette) (% w/v) | | | | | | | | |
| (Average MS: 0.62, MS | | | | | | | | |
| range: 0.58-0.68)¹ | | | | | | | | |
| HP-B-CD | — | — | — | — | — | — | — | 22 |
| KLEPTOSE ® | | | | | | | | |
| (Roquette) (% w/v) | | | | | | | | |
| (Average MS: 0.92, MS | | | | | | | | |
| range: 0.81-0.99)¹ | | | | | | | | |
| L-Histidine (mM) | 6 | 6 | 6 | 6 | 6 | — | — | — |
| Citrate, 1 H₂O (mM) | — | — | — | — | — | 3 | 3 | 3 |
| Sorbitol (mg/ml) | 20 | 20 | 20 | 35 | 20 | — | — | — |
| Polysorbate 80 (mg/ml) | 0.05 | 0.05 | 0.05 | 0.05 | 0.05 | 0.05 | 0.05 | 0.05 |
| HCl | q.s. | q.s. | q.s. | q.s. | q.s. | q.s. | q.s. | q.s. |
| NaOH | q.s. | q.s. | q.s. | q.s. | q.s. | q.s. | q.s. | q.s. |
| WFI | To 1 | To 1 | To 1 | To 1 | To 1 | To 1 | To 1 | To 1 |
| | ml | ml | ml | ml | ml | ml | ml | ml |
| pH | 6.0 | 6.0 | 6.0 | 6.0 | 6.0 | 6.0 | 6.0 | 6.0 |

¹MS: molar substitution, corresponds to hydroxypropyls per glucose unit

### Preparation Process

Formulations were prepared as described in example 1 except that active pharmaceutical ingredients were not added.

### Method

The local (subcutaneous) tolerance, upon subcutaneous administration, of formulations containing HP-B-CD was studied in 4 live Landrace×Yorkshire×Duroc (LYD) pigs by evaluation of the resulting skin lesions 5 days (necropsy) after subcutaneous administration of 200 μl using NovoPen 4 with NovoFine Plus needles (32 G/4 mm). Skin samples sized 2×2 cm were collected at necropsy, fixed in neutral buffered formalin, trimmed using multi-knife, embedded in paraffin, cut in 4 μm thin sections, mounted on glass slides and subsequently hematoxylin-eosin (HE) stained. For the four samples, the severity of the subcutaneous tissue necrosis and inflammatory cell infiltration was assessed by a trained toxicopathologist using a light microscope and scored on a numerical scale, where code 1 reflects ‘no abnormality detected’ and code 5 reflects ‘marked severity’:
1, no abnormality detected
2, minimal severity
3, mild severity
4, moderate severity
5, marked severity

The co-formulation vehicles were evaluated for the level of subcutaneous tissue necrosis and inflammatory cell infiltration that they elicited upon subcutaneous injection. The results are presented in table 47.

TABLE 47
Severity scores: severity of subcutaneous tissue necrosis and
inflammatory cell infiltration 5 days post injection, for co-formulation
vehicles varying in HP-B-CD content and molar substitution degree as
well as in overall buffer composition

| | | Inflammatory cell |
|---|---|---|
| | | infiltration, |
| Co-formulation vehicle | Necrosis | granulomatous |
| 9 | 1, 1, 1, 1 | 2, 2, 2, 2 |
| 10 | 1, 1, 2, 2 | 2, 2, 2, 3 |
| 11 | 2, 3, 4, 4 | 3, 3, 4, 4 |
| 12 | 2, 4, 4, 4 | 3, 3, 4, 4 |
| 13 | 3, 4, 4, 4 | 3, 3, 4, 5 |
| 14 | 1, 3, 3, 3 | 1, 3, 3, 3 |
| 15 | 3, 4, 5, 5 | 4, 4, 4, 5 |
| 16 | 1, 5, 4, 5 | 2, 4, 4, 5 |

### Concluding Remarks

The results in table 47 show that the in vivo local subcutaneous tolerability depended on the concentration of HP-B-CD and the overall buffer composition. Co-formulation vehicles comprising histidine and sorbitol showed better tolerability than those comprising citrate.

Co-formulation vehicles comprising 20% w/v HP-B-CD or less resulted mainly in no or minimal necrosis or inflammatory cell infiltration (scores of 1 or 2) and a single observation of mild inflammatory cell infiltration (a score of 3). Co-formulation vehicles comprising 22% w/v HP-B-CD or more all resulted in minimal to moderate necrosis and inflammatory cell infiltration (scores of up to 4). Based on these results, co-formulations containing less than 22% HP-B-CD appear suitable for subcutaneous use.

The co-formulation vehicles containing 20% w/v and 22% w/v HP-B-CD and citrate (co-formulation vehicles 14 and 15) resulted in marked necrosis and inflammatory cell infiltration (scores of up to 5). Surprisingly, co-formulation vehicles containing 20% w/v and 22% w/v HP-B-CD, histidine and sorbitol (co-formulation vehicles 10 and 11) were more well tolerated, resulting in moderate necrosis and inflammatory cell infiltration (scores of up to 4).

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A liquid pharmaceutical formulation comprising an amylin receptor agonist, a GLP-1 receptor agonist and a cyclodextrin of the hydroxypropyl-substituted alpha and/or beta type and having a pH of 5.5-6.5.

2. The pharmaceutical formulation according to claim 1, wherein said GLP-1 receptor agonist is semaglutide.

3. The pharmaceutical formulation according to claim 1, wherein said amylin receptor agonist is cagrilintide.

4. The pharmaceutical formulation according to claim 1, wherein said cyclodextrin is of the hydroxypropyl-substituted beta type.

5. The pharmaceutical formulation according to claim 1 4, wherein said cyclodextrin comprises a minimum of about 0.4 hydroxypropyls per glucose unit and a maximum of about 1.0 hydroxypropyls per glucose unit.

6. The pharmaceutical formulation according to claim 1, comprising more than 10% w/v and less than 22% w/v cyclodextrin.

7. The pharmaceutical formulation according to claim 1, comprising an effective amount of cagrilintide and semaglutide.

8. The pharmaceutical formulation according to claim 1, further comprising a tonicity agent, with the proviso that the tonicity agent is not sodium chloride.

9. The pharmaceutical formulation according to claim 8, wherein said tonicity agent is sorbitol, mannitol and/or trehalose.

10. The pharmaceutical formulation according to claim 1, further comprising a buffer having at least one pKa of about 5.0-7.0, such as citrate and/or histidine; preferably, histidine.

11. The pharmaceutical formulation according to claim 1, further comprising a surfactant.

12. A pharmaceutical formulation comprising:
an effective amount of cagrilintide and semaglutide,
more than 10% w/v and less than 22% w/v cyclodextrin of the hydroxypropyl-substituted alpha and/or beta type, said cyclodextrin comprising a minimum of about 0.4 and a maximum of about 1.0 hydroxypropyls per glucose unit,
about 3-30 mM histidine,
about 10-40 mg/ml sorbitol,
up to 2.0 mg/ml, polysorbate 20 and/or 80,
about 75-90% w/w water and:
having a pH of 5.6-6.0.

13. (canceled)

14. (canceled)

15. (canceled)

16. The pharmaceutical formulation according to claim 12, wherein said cyclodextrin comprising an average of 0.62-0.84 hydroxypropyls per glucose unit.

17. A pharmaceutical formulation comprising: an effective amount of cagrilintide and semaglutide, 10-20% w/v cyclodextrin of the hydroxypropyl-substituted alpha and/or beta type, said cyclodextrin comprising 0.58-1.0 hydroxypropyls per glucose unit, about 3-30 mM histidine, about 10-40 mg/ml sorbitol, up to 1.5 mg/ml polysorbate 20 and/or 80, about 75-90% w/w water and having a pH of 5.6-6.0.

18. The pharmaceutical formulation according to claim 16, wherein said cyclodextrin comprising an average of 0.62-0.84 hydroxypropyls per glucose unit.

19. A method of treating overweight or obesity, with or without one or more associated co-morbidities; diabetes, with or without one or more associated comorbidities; one or more cardiovascular diseases; non-alcoholic steatohepatitis (NASH); or cognitive impairment, comprising administering a pharmaceutical formulation according to claim 12.

20. A method of treating overweight or obesity, with or without one or more associated co-morbidities; diabetes, with or without one or more associated comorbidities; one or more cardiovascular diseases; non-alcoholic steatohepatitis (NASH); or cognitive impairment, comprising administering a pharmaceutical formulation according to claim 18.

21. The pharmaceutical formulation according to claim 7, wherein said cyclodextrin is of the hydroxypropyl-substituted beta type.

22. The pharmaceutical formulation according to claim 7, wherein said cyclodextrin comprises a minimum of about 0.4 hydroxypropyls per glucose unit and a maximum of about 1.0 hydroxypropyls per glucose unit.

23. The pharmaceutical formulation according to claim 7, comprising about 15% w/v cyclodextrin.

24. The pharmaceutical formulation according to claim 7, further comprising a tonicity agent, with the proviso that the tonicity agent is not sodium chloride.

25. The pharmaceutical formulation according to claim 24, wherein said tonicity agent is sorbitol.

26. The pharmaceutical formulation according to claim 7, further comprising a histidine buffer or a citrate buffer.

27. The pharmaceutical formulation according to claim 7, further comprising polysorbate 20 and/or polysorbate 80.
